SEQUENCE LISTING (38 sequences)

SEQ ID NO: 1 - DNA, 73 bp, Artificial Sequence
  Synthetic oligonucleotide
gtagatcatatcaccaatcgattctacagtatcgtaatataacatgactctcgctcaactatcgatacagtgc

SEQ ID NO: 2 - DNA, 73 bp, Artificial Sequence
  Synthetic oligonucleotide
guautgtatugatagttgagugagagtuatgttatattaugatautgtagaatagattggtgatatgatutau

SEQ ID NO: 3 - DNA, 73 bp, Artificial Sequence
  Synthetic oligonucleotide
gtagatuatatuauuaatugattutauagtatugtaatataauatgaututugutuaautatugatauagtgu

SEQ ID NO: 4 - DNA, 24 bp, Artificial Sequence
  Synthetic oligonucleotide
acactatatcaataattaaacaaa

SEQ ID NO: 5 - DNA, 73 bp, Artificial Sequence
  Synthetic oligonucleotide
acactatatcaataattaaacaaaaatcatattatattacaatactataaaatcaattaataatataatctac

SEQ ID NO: 6 - DNA, 24 bp, Artificial Sequence
  Synthetic oligonucleotide
gtagattatattatcaattgattt

SEQ ID NO: 7 - DNA, 73 bp, Artificial Sequence
  Synthetic oligonucleotide
acactatatcaataattaaacaaaaatcatattatattacaatactataaaatcaattgataatataatctac

SEQ ID NO: 8 - DNA, 73 bp, Artificial Sequence
  Synthetic oligonucleotide
gtagattatattatcaattgattttatagtattgtaatataatatgattttagtttaattattgatatagtgt

SEQ ID NO: 9 - DNA, 24 bp, Artificial Sequence
  Synthetic oligonucleotide
tatagtatcgtaatataatatgat

SEQ ID NO: 10 - DNA, 22 bp, Artificial Sequence
  Synthetic oligonucleotide
caatcgattctacagtattgta

SEQ ID NO: 11 - DNA, 54 bp, Artificial Sequence
  Synthetic oligonucleotide
tagctgtatcgtcaaggcactcttgcctacgccaccagctccaactaccacaag

SEQ ID NO: 12 - DNA, 22 bp, Artificial Sequence
  Synthetic oligonucleotide
acgccacaagctccaactacca

SEQ ID NO: 13 - DNA, 21 bp, Artificial Sequence
  Synthetic oligonucleotide
acgccacgagctccaactacc

SEQ ID NO: 14 - DNA, 23 bp, Artificial Sequence
  Synthetic oligonucleotide
acgccactagctccaactaccac

SEQ ID NO: 15 - DNA, 22 bp, Artificial Sequence
  Synthetic oligonucleotide
acgccaacagctccaactacca

SEQ ID NO: 16 - DNA, 21 bp, Artificial Sequence
  Synthetic oligonucleotide
acgccagcagctccaactacc

SEQ ID NO: 17 - DNA, 23 bp, Artificial Sequence
  Synthetic oligonucleotide
acgccatcagctccaactaccac

SEQ ID NO: 18 - DNA, 23 bp, Artificial Sequence
  Synthetic oligonucleotide
acgacaccagctccaactaccac

SEQ ID NO: 19 - DNA, 22 bp, Artificial Sequence
  Synthetic oligonucleotide
actcttgcctacgccaccagct

SEQ ID NO: 20 - DNA, 24 bp, Artificial Sequence
  Synthetic oligonucleotide
ctcttgcctacgccaccagctcca

SEQ ID NO: 21 - DNA, 27 bp, Artificial Sequence
  Synthetic oligonucleotide
gcactcttgcctacgccaccagctcca

SEQ ID NO: 22 - DNA, 27 bp, Artificial Sequence
  Synthetic oligonucleotide
cgaccaccgcatccgttctcacggaac

SEQ ID NO: 23 - DNA, 22 bp, Artificial Sequence
  Synthetic oligonucleotide
tatcgtcaaggcactcttgcct

SEQ ID NO: 24 - DNA, 22 bp, Artificial Sequence
  Synthetic oligonucleotide
agctgtatcgtcaaggcactct

SEQ ID NO: 25 - DNA, 21 bp, Artificial Sequence
  Synthetic oligonucleotide
ctgtatcgtcaaggcactctt

SEQ ID NO: 26 - DNA, 36 bp, Homosapien
  promoter (0)...(0): DAPK-1 promoter
actcggcaactcgcagcggcagggtctggggccggc

SEQ ID NO: 27 - DNA, 36 bp, Homosapien
  promoter (0)...(0): DAPK-1 promoter
autugguaautuguagugguagggtutgggguuggu

SEQ ID NO: 28 - DNA, 36 bp, Homosapien
  promoter (0)...(0): DAPK-1 promoter
accaaccccaaaccctaccactacaaattaccaaat

SEQ ID NO: 29 - DNA, 36 bp, Homosapien
  promoter (0)...(0): DAPK-1 promoter
autcgguaautcguagcgguagggtutggggucggc

SEQ ID NO: 30 - DNA, 36 bp, Homosapien; identical to SEQ ID NO: 28
  promoter (0)...(0): DAPK-1 promoter

SEQ ID NO: 31 - DNA, 22 bp, Artificial Sequence
  Synthetic oligonucleotide
ggatagttggattctgttaatg

SEQ ID NO: 32 - DNA, 19 bp, Artificial Sequence
  Synthetic oligonucleotide
ggagtgtgaggaggatagt

SEQ ID NO: 33 - DNA, 19 bp, Artificial Sequence
  Synthetic oligonucleotide
ggatcctgttaacgttggg

SEQ ID NO: 34 - DNA, 22 bp, Artificial Sequence
  Synthetic oligonucleotide
cctccgcaaaaaaaacaaaatc

SEQ ID NO: 35 - DNA, 61 bp, Homosapien
  promoter (0)...(0): DAPK-1 promoter
ggagtgtgaggaggauaguuggauugaguuaauguuggggautttgttuuutuuguggagg

SEQ ID NO: 36 - DNA, 61 bp, Homosapien
  promoter (0)...(0): DAPK-1 promoter
cctcacactcctcctatcaacctaactcaattacaacccctaaaacaaaaaaaacacctcc

SEQ ID NO: 37 - DNA, 61 bp, Homosapien
  promoter (0)...(0): DAPK-1 promoter
ggagtgtgaggaggauagucggaucgaguuaacguuggggautttgttuuutuugcggagg

SEQ ID NO: 38 - DNA, 61 bp, Homosapien
  promoter (0)...(0): DAPK-1 promoter
cctccgcaaaaaaaacaaaatccccaacgttaactcgatccgactatcctcctcacactcc

(19) United States
(12) Patent Application Publication
(10) Pub. No.: US 2015/0315636 A1
(43) Pub. Date: Nov. 5, 2015

(54) SELECTIVE AMPLIFICATION AND REAL-TIME PCR DETECTION OF RARE MUTATIONS

(71) Applicant: BECTON, DICKINSON AND COMPANY, Franklin Lakes, NJ (US)

(72) Inventors: James G. Nadeau, Ellicott City, MD (US); Tobin J. Hellyer, Westminster, MD (US)

(21) Appl. No.: 14/439,634

(22) PCT Filed: Oct. 30, 2013
(86) PCT No.: PCT/US13/67604

§ 371 Date: Apr. 29, 2015

Related U.S. Application Data
(60) Provisional application No. 61/720,959, filed on Oct. 31, 2012.

(51) Int. Cl. C12Q 1/68

(52) U.S. Cl. CPC C12Q 1/6858

(57) ABSTRACT

Provided herein are methods and kits for the improved detection of rare mutations within a high background. Exemplary embodiments relate to kits and methods that include amplification primers, a blocking oligonucleotide, and one or more allele-specific detector probes, useful in the specific detection of rare allelic variants or mutations.

### CROSS-REFERENCE TO RELATED APPLICATIONS

This application claims priority to U.S. Provisional Patent Application No. 61/720,959, entitled “SELECTIVE AMPLIFICATION AND REAL-TIME PCR DETECTION OF RARE MUTATIONS,” filed Oct. 31, 2012, the entire content of which is hereby incorporated by reference.

### REFERENCE TO SEQUENCE LISTING, TABLE, OR COMPUTER PROGRAM LISTING

The present application is being filed along with a Sequence Listing in electronic format. The Sequence Listing is provided as a file entitled GENOM122.txt, last saved Oct. 30, 2013, which is 7.66 kb in size. The information is incorporated herein by reference in its entirety.

### BACKGROUND OF THE INVENTION

1. Field of the Invention

The present embodiments relate to molecular diagnostics, and in particular, to compositions in detecting sequence variants, such as SNPs, insertions deletions, and altered methylation patterns, from samples. The embodiments disclosed herein can be used to detect (and quantify) sequence variants present in samples that include an excess of wild-type sequences.

2. Description of the Related Art

With the advent of molecular diagnostics and the discovery of numerous nucleic acid biomarkers useful in the diagnosis and treatment of conditions and diseases, detection of nucleic acid sequences, and sequence variants, mutations and polymorphisms has become increasingly important. In many instances, it is desirable to detect sequence variants or mutations (which may in some instances, differ by one a single nucleotide) present in low copy numbers against a high background of wild-type sequences. For example, as more and more somatic mutations are shown to be biomarkers for cancer prognosis and prediction of therapeutic efficacy, the need for efficient and effective methods to detect rare mutations in a sample is becoming more and more critical.

In the case in which one or more allelic variants is/are present in low copy number compared to wild-type sequences, the presence of excess wild-type target sequence creates challenges to the detection of the less abundant variant target sequence. Nucleic acid amplification/detection reactions almost always are performed using limiting amounts of reagents. A large excess of wild-type target sequences, thus competes for and consumes limiting reagents. As a result amplification and/or detection of rare mutant or variant alleles under these conditions is substantially suppressed, and the methods may not be sensitive enough to detect the rare variants or mutants. Various methods to overcome this problem have been attempted. These methods are not ideal, however, because they either require the use of a unique primer for each allele, or the performance of an intricate melt-curve analysis. Both of these shortcomings limit the ability and feasibility of multiplex detection of multiple variant alleles from a single sample.

### SUMMARY OF THE INVENTION

Detection of rare sequence variants in biological samples presents numerous challenges. The methods and kits disclosed herein provide for improved, efficient means to detect rare mutations within a high background of wild-type allelic sequences using real-time amplification methods.

In one aspect, the embodiments disclosed herein relate to methods to detect a first variant target sequence in a sample comprising nucleic acids. The method can include the steps of providing the biological sample for analysis, and contacting the biological sample with a pair of amplification primers. The amplification primers can include a forward primer and a reverse primer which together are configured to amplify a target amplicon or target region. The target amplicon, or target region, can include either a wild-type target allele sequence or a variant target allele sequence of interest. The amplification primers can flank the wild-type target sequence or variant target allele sequence, such that the amplification primers amplify both wild-type target sequences and variant target allele sequences under amplification or primer extension conditions. The sample can also be contacted with a blocking primer that preferentially hybridizes to the wild type target allele sequence compared to a first variant target allele sequence under amplification conditions. The sample can also be contacted with one or more reporter probes, wherein the reporter probe(s) include an oligonucleotide that preferentially hybridizes to the first variant target allele sequence compared to the wild-type target sequence under amplification conditions, and a detectable moiety. The sample can be contacted with the amplification primers, the blocking oligonucleotide and the detector probe(s) under amplification conditions. Hybridization of the reporter probe to the first variant target allele sequence can be measured, wherein hybridization of the reporter probe to the first variant target allele sequence produces a detectable signal indicative of the presence and/or amount of first variant target allele species in the biological sample.

In another aspect, the embodiments disclosed herein provide kits and compositions for the detection of a rare sequence variant or mutant allele from a sample. The kits or compositions can include an amplification primer pair that includes a forward and reverse primer that flank a target region, that includes within the target region the target variant or mutant allele sequence of interest. The kits and compositions can also include a blocking oligonucleotide that is non-extendible by a polymerase, and which preferentially hybridizes to a wild type target allele sequence compared to the variant or mutant target allele sequence. The kits and compositions can also include a detector probe. The detector probe can include an oligonucleotide that preferentially binds to the variant or mutant target allele sequence compared to the wild-type target allele sequence. The detector probe also includes a detectable moiety that enables detection of hybridization of the detector probe to the variant or mutant target allele sequence.

### BRIEF DESCRIPTION OF THE DRAWINGS

FIG. 1 is a schematic of an exemplary embodiment illustrating a method for detection of a single, rare variant allele according to the embodiments disclosed herein.

FIG. 2 is a schematic of an exemplary embodiment illustrating a method for the simultaneous detection of more than one rare, variant allele according to the embodiments disclosed herein.

FIG. 3 is a schematic of an exemplary embodiment illustrating a method for the detection of methylation variants according to the embodiments disclosed herein.

FIGS. 4A-D is a schematic showing the different, possible species of molecular complexes in in reaction mixtures containing an analyte (A), amplification primer (P), blocking oligonucleotide (B), detector probe (D) and polymerase (E).

FIG. 5 shows the equilibrium between the various species of molecular complexes shown in FIGS. 4A-D.

FIG. 6 illustrates an exemplary method to estimate the fraction extendible target species, i.e., “fe.,” of the complexes shown in FIG. 4, according to the embodiments disclosed herein

FIG. 7 shows a mathematical model for an amplification reaction on a sample comprising two different target species, according to the embodiments disclosed herein.

FIG. 8 depicts the various reporter probes blocking oligonucleotides and forward amplification primers used in the simulated real-time PCR assays discussed in EXAMPLE 1.

FIGS. 9A-B show simulated amplification curves of real-time amplification reactions using the various conditions described in EXAMPLE 1, with a mixture of wild-type and G34T mutant KRAS nucleic acids, present in a ratio of 10000:100 (wt:mutant). FIG. 9A shows the amplification curve (relative fluorescence v. cycle number) of the reaction under the described parameters, wherein the W.T. fe., as explained in EXAMPLE 1, is approximately 0.159. FIG. 9B shows the amplification curve (relative fluorescence v. cycle number) of the reaction under the described, wherein the WT f.e. is approximately 0.717, as described in EXAMPLE 1.

FIG. 10A depicts a target region of the DAPK-1 promoter region as described in EXAMPLE 2, including the location of cytosine residues that are potentially methylated. CpG sites are boxed.

FIG. 10B depicts a schematic showing a reaction to detect methylation variants in the DAPK-1 promoter, as described in EXAMPLE 2.

### DETAILED DESCRIPTION OF THE PREFERRED EMBODIMENT

It is to be understood that both the foregoing general description and the following detailed description are exemplary and explanatory only and are not intended to limit the scope of the current teachings. In this application, the use of the singular includes the plural unless specifically stated otherwise. Also, the use of “comprise”, “contain”, and “include”, or modifications of those root words, for example but not limited to, “comprises”, “contained”, and “including”, are not intended to be limiting. Use of “or” means “and/or” unless stated otherwise. The term “and/or” means that the terms before and after can be taken together or separately. For illustration purposes, but not as a limitation, “X and/or Y” can mean “X” or “Y” or “X and Y”.

Whenever a range of values is provided herein, the range is meant to include the starting value and the ending value and any value or value range there between unless otherwise specifically stated. For example, “from 0.2 to 0.5” means 0.2, 0.3, 0.4, 0.5; ranges there between such as 0.2-0.3, 0.3-0.4, 0.2-0.4; increments there between such as 0.25, 0.35, 0.225, 0.335, 0.49; increment ranges there between such as 0.26-0.39; and the like.

The section headings used herein are for organizational purposes only and are not to be construed as limiting the subject matter described in any way. All literature and similar materials cited in this application including, but not limited to, patents, patent applications, articles, books, treatises, and internet web pages, regardless of the format of such literature and similar materials, are expressly incorporated by reference in their entirety for any purpose. In the event that one or more of the incorporated literature and similar materials defines or uses a term in such a way that it contradicts that term's definition in this application, this application controls. While the present teachings are described in conjunction with various embodiments, it is not intended that the present teachings be limited to such embodiments. On the contrary, the present teachings encompass various alternatives, modifications, and equivalents, as will be appreciated by those of skill in the art.

The embodiments disclosed herein provide improved methods for detection of mutant or variant alleles. The methods disclosed herein advantageously overcome many of the limitations of previous methods of molecular detection of rare mutations, and enable detection of multiple alleles within a single real-time PCR reaction, without the requirement for multiple, allele-specific amplification primers.

### Detection of Variant or Mutant Alleles

Provided herein are methods for analyzing a sample for allelic variants within a target sequence. Allelic variants have been implicated in genetic disorders, susceptibility to different diseases, responses to various therapeutics and the like. Accordingly, the importance of detection of allelic variants or mutations in target sequences cannot be underestimated. As used herein, the term “target sequence” refers to a nucleic acid sequence of interest, e.g., a genomic DNA, an mRNA, a cDNA, or the like, to be queried for the presence of allelic variants, e.g., rare allelic variants or mutations. As used herein, the term “rare allelic variant” or “variant target sequence,” refers to a target sequence that is present at a lower copy number in a sample compared to an alternative allelic variant, such as a wild-type target sequence. For example, the variant target sequence may be present in a sample at a frequency of less than 1/10, 1/100, 1/1,000, 1/10,000, 1/100,000, 1/1,000,000, 1/10,000,000, 1/100,000,000, 1/1,000,000,000, or less (or any frequency in between), compared to another allelic variant or wild-type target sequence. For example, a rare allelic variant or variant target sequence, may be present at less than 2, 3, 4, 5, 6, 7, 8, 9, 10, 20, 30, 40, 50, 60, 70, 80, 90, 100, 200, 300, 500, 750, 1000, 2500, 5000, 7500, 10000, 25000, 50000, 75000, 100000, 250000, 500000, 750000, 1000000, or more, copies in a sample. Insome embodiments, the term allelic variant can refer to single nucleotide polymorphisms, substitutions, insertions, deletions, or the like.

The methods disclosed herein can be used in the detection of numerous allelic variants, including nonsense mutations, missense mutations, insertions, deletions, and the like. Owing to the advantageous sensitivity and specificity of detection afforded by the methods disclosed herein, the methods can detect the presence of a rare allelic variant within a sample, amongst a high wild-type background. Accordingly, although the skilled artisan will appreciate that the methods disclosed herein can be used in a variety of settings to detect, e.g., germline mutations, the methods are particularly well-suited for use in the detection of somatic mutations, such as mutations present in tumors. Non-limiting examples of rare, somatic mutations useful in the diagnosis, prognosis, and treatment of various tumors include, for example, mutations in ABL, AKT1, AKT2, ALK, APC, ATM, BRAF, CBL, CDH1, CDKN2A, CEBPA, CRLF2, CSF1R, CTNNB1, EGFR, ERBB2, EZH2,FBXW7, FGFR, FGFR2, FGFR3, FLT3, FOXL2, GATA1, GATA2, GNAQ, GNAS, HNF1A, HRAS, IDH1, IDH3, JAK2, KIT, KRAS, MEK1, MET, MPL, NF2, NOTCH1, NOTCH2, NPM, NRAS, PCA3, PDGFRA, PIK3CA, PIK3R1, PIK3R5, PTCH1, PTEN, PTPN11, RB1, RET, RUNX1, SMAD4, SMARCB, SMO, STK11, TET2, P53, TSHR, VHL, WT1, and others. Exemplary mutant alleles associated with cancer useful in the embodiments disclosed herein include, but are not limited to those described in publications listed on the world wide web site for COSMIC (Catalogue Of Somatic Mutations In Cancer) available at sanger.ac.uk/genetics/CGP/cosmic/add info. Exemplary mutations are listed in Table 1, annexed hereto.

DNA methylation is an important mechanism of epigenetic gene regulation. Rare changes in the DNA methylation patterns of genes associated with cell growth and differentiation have been linked to a variety of cancers. As such, detection of rare, altered DNA methylation patterns offers potential in cancer diagnosis, treatment and therapeutic monitoring. By way of example, epigenetic silencing of tumor suppressor genes through hypermethylation of their promoter regions is frequently associated with the onset of disease and detection of such changes may have utility in early diagnosis. Accordingly, in some embodiments, the methods disclosed herein can be advantageously used to detect rare, altered DNA methylation patterns, e.g., to enhance the specificity of detection of low levels of DNA methylation in a background of high levels of unmethylated DNA, to enhance the sensitivity and specificity of detection of rare methylation events, and/or to enhance the detection of unmethylated DNA or loss of methylation in a background of highly methylated DNA. Non-limiting examples of variations in DNA methylation that can be advantageously queried using the methods described herein include, but are not limited to the detection of methylation of the promoter region of Human Death Associated Kinase Protein-1 (DAKP1) gene, promoter in genes involved in cell cycle, growth differentiation and development (e.g., BRCA1, CCNA, CCND2, CDKN1C, CDKN2A (p14ARF), CDKN2A (p16), SFN, TP73, and the like), cell adhesion genes, e.g., CDH1, CDH13, OPCML (aOBCAM), PCDH10 and the like; transcription factors, e.g., ESR1, HIC1, PRDM2, RASSF1, TP73, HIC1, HNF1B, RUNX3, WT1.; hormone receptors, e.g., ESR1; drug metabolism genes, e.g., GSTP1, and the like; genes involved in apoptosis and anti-apoptosis, e.g., PYCARD, TNFRSF10C, TNFRSF10D, APC and the like, phosphatases, e.g., PTEN, DNA methylation, e.g., MGMT, PRDM2; extracellular matrix molecules, e.g., ADAM23, SLIT2, THBS1, as well as other genes, e.g., RASSF1, and the like; miRNAs, e.g., let-7g, mir-10a, mir-124-2, mir-126, mir-149, mir-155, mir-15b Cluster (mir-15b, mir-16-2), mir-17 cluster (mir-17, mir-18a, mir-19a, mir-19b-1, mir-20a, mir-92a-1), miR-191 Cluster (miR-191, miR-425), mir-210, mir-218-1, mir-218-2, mir-23b Cluster (mir-23b, mir-24-1, mir-27b), mir-301a, mir-30c-1 Cluster (mir-30c-1, mir-30e), mir-32, mir-378, mir-7-1, and the like.

The methods disclosed herein can be used to analyze nucleic acids of samples. The term “sample” as described herein can include bodily fluids (including, but not limited to, blood, urine, feces, serum, lymph, saliva, anal and vaginal secretions, perspiration, peritoneal fluid, pleural fluid, effusions, ascites, and purulent secretions, lavage fluids, drained fluids, brush cytology specimens, biopsy tissue (e.g., tumor samples), explanted medical devices, infected catheters, pus, biofilms and semen) of virtually any organism, with mammalian samples, particularly human samples.

In some embodiments, the sample is processed prior to the nucleic acid testing. For example, in some embodiments, the sample is processed to extract and/or separate and/or isolate nucleic acids from other material present in the sample. In some embodiments, the sample is analyzed directly, e.g., without prior nucleic acid extraction and/or isolation. In some embodiments, the sample is processed in order to isolate genomic DNA. In some embodiments, the sample is processed in order to isolate mRNA. In some embodiments, the sample is processed by using RT-PCR to generate cDNA, prior to the nucleic acid testing. Methods for processing samples and nucleic acids in accordance with the methods disclosed herein are well-known, and are described, e.g., in Current Protocols in Molecular Biology, Greene Publ. Assoc. Inc. & John Wiley & Sons, Inc., NY, N.Y.; Sambrook et al. (1989) Molecular Cloning, Second Ed., Cold Spring Harbor Laboratory, Plainview, N.Y.); Maniatis et al. (1982) Molecular Cloning, Cold Spring Harbor Laboratory, Plainview, N.Y.; and elsewhere.

### Detection of Sequence Variants

Provided herein are methods useful in the detection of sequence variants, i.e., insertions, deletions, nonsense mutations, missense mutations, and the like. In the methods for detecting allelic variants or variant target sequences disclosed herein, the sample, which comprises the nucleic acids to be analyzed, are contacted with an amplification primer pair, i.e., comprising a forward primer and a reverse primer that flank the target sequence or target region containing a sequence of interest (e.g., a wild-type, mutant, or variant allele sequence) to be analyzed. By “flanking” the target sequence, it is understood that the variant or wild-type allelic sequence is located between the forward and reverse primers, and that the binding site of neither the forward nor reverse primer comprises the variant or wild-type allelic sequence to be assessed. For example, in some embodiments, the variant or wild-type allelic sequence to be assessed is removed from or positioned away from the 3′ end of either oligonucleotide by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more, e.g., 100 or more, 200 or more, 300 or more, 400 or more, 500 or more, etc., nucleotides. Amplification primers that flank, but that do not overlap with, the variant target sequence or the wild-type target sequence are thus not “allele-specific” amplification primers, and are capable of amplification of various different alleles or variants of a sequence of interest. Thus, in some embodiments, the amplification primers are configured to amplify various mutant or variant alleles and wild type alleles non-preferentially. As discussed in further detail below, the addition of blocking oligonucleotides to an amplification reaction suppresses the amplification of wild-type target sequences and enables preferential amplification of non-wild-type, e.g., variant, mutant or rare variant alleles.

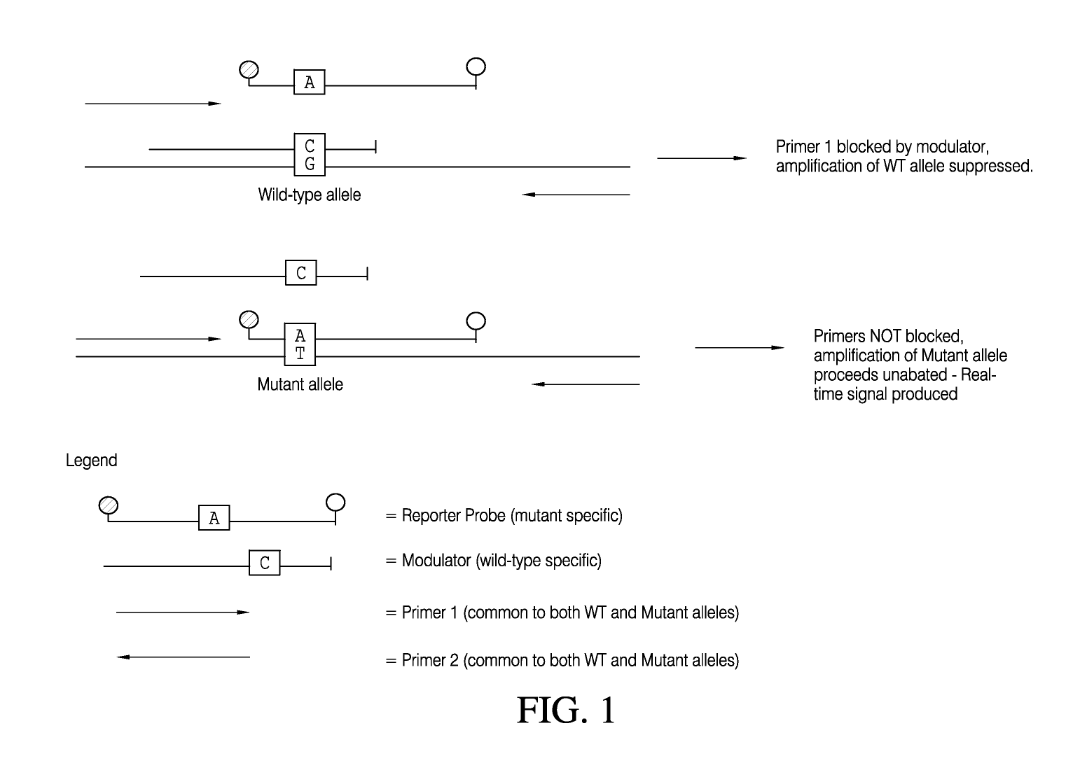

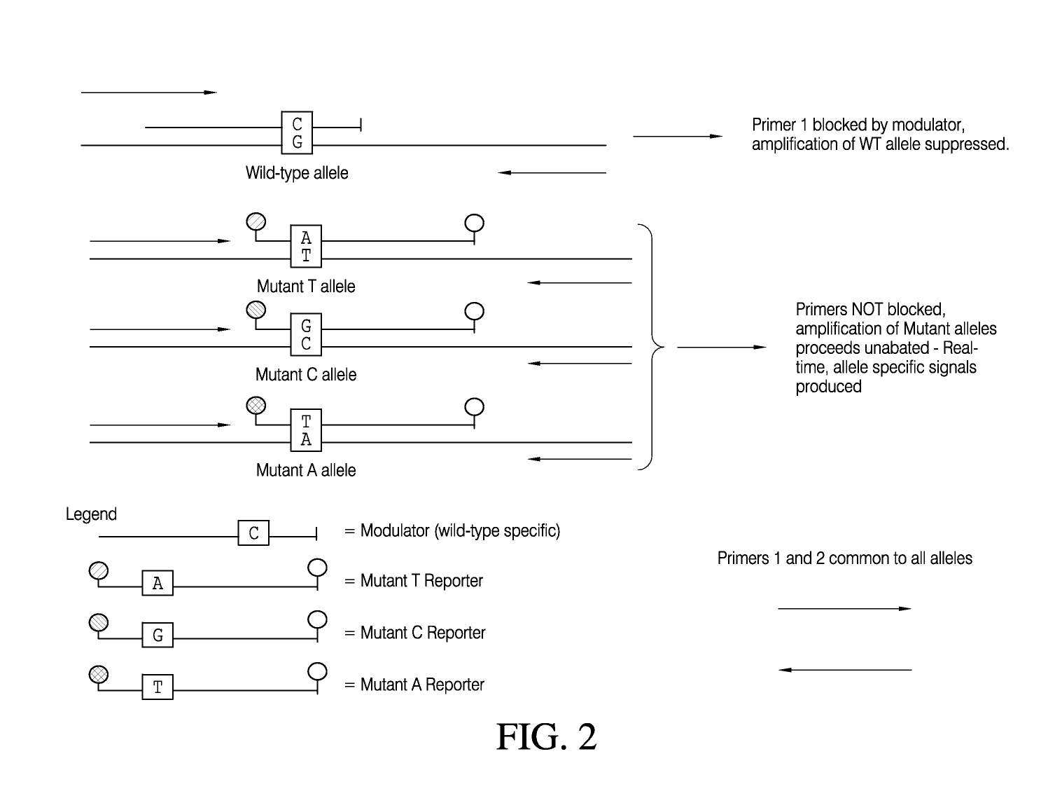

FIGS. 1 and 2 are depictions of exemplary methods according to the embodiments disclosed herein for the detection of sequence variants. As shown in FIGS. 1 and 2, amplification primers (i.e., forward primer 1 and reverse primer 2) flank the wild type and mutant allele sequences of interest, and comprise sequences common to both wild-type and mutant or variant allele sequences. Accordingly, as shown in FIG. 2, in contrast to methods that utilize allele-specific amplification primers to achieve preferential amplification of rare sequences, the present methods advantageously enable the simultaneous amplification of multiple variant sequences, using a single amplification primer pair.

### Detection of Altered DNA Methvlation Patterns

Also provided are methods for the detection of DNA methylation variants, i.e., DNA that has an altered methylation pattern—e.g., is methylated at cytosine residues that are non-methylated in wild-type DNA, or includes unmethylated cytosine residues that are methylated in wild-type DNA.

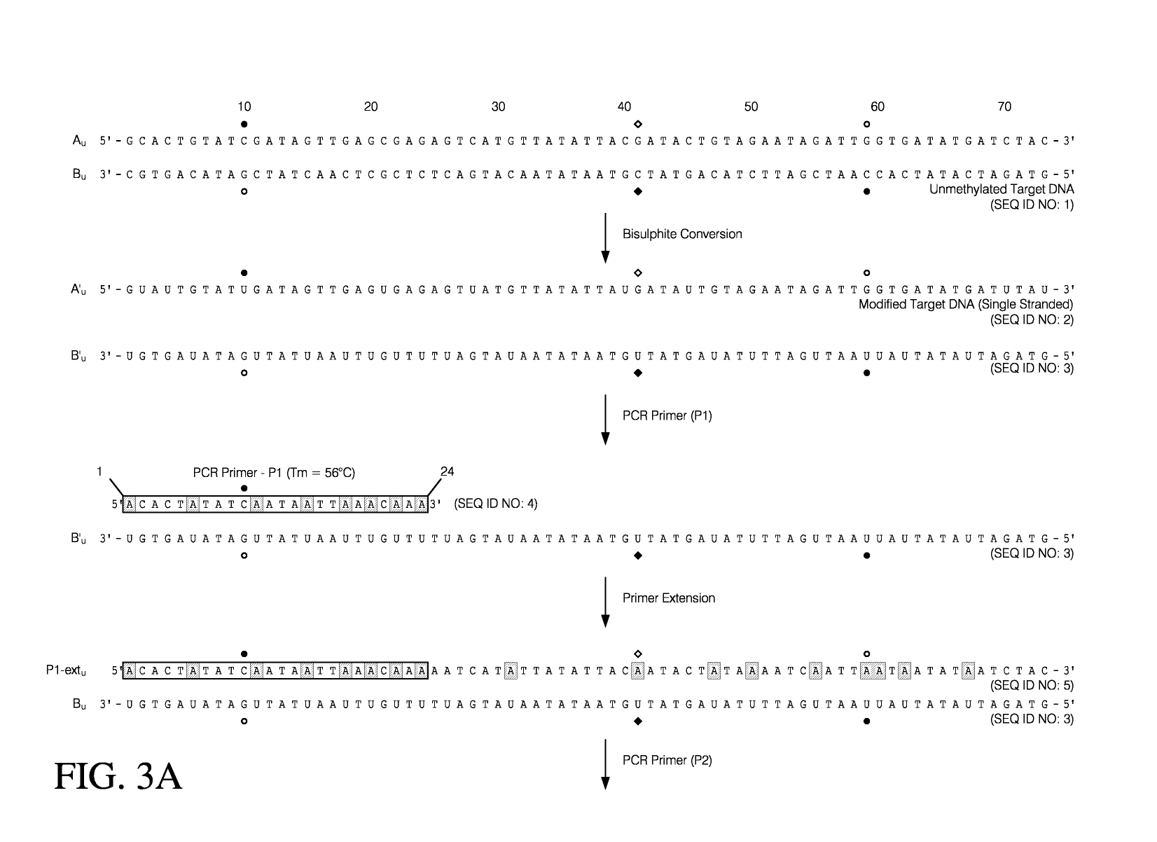

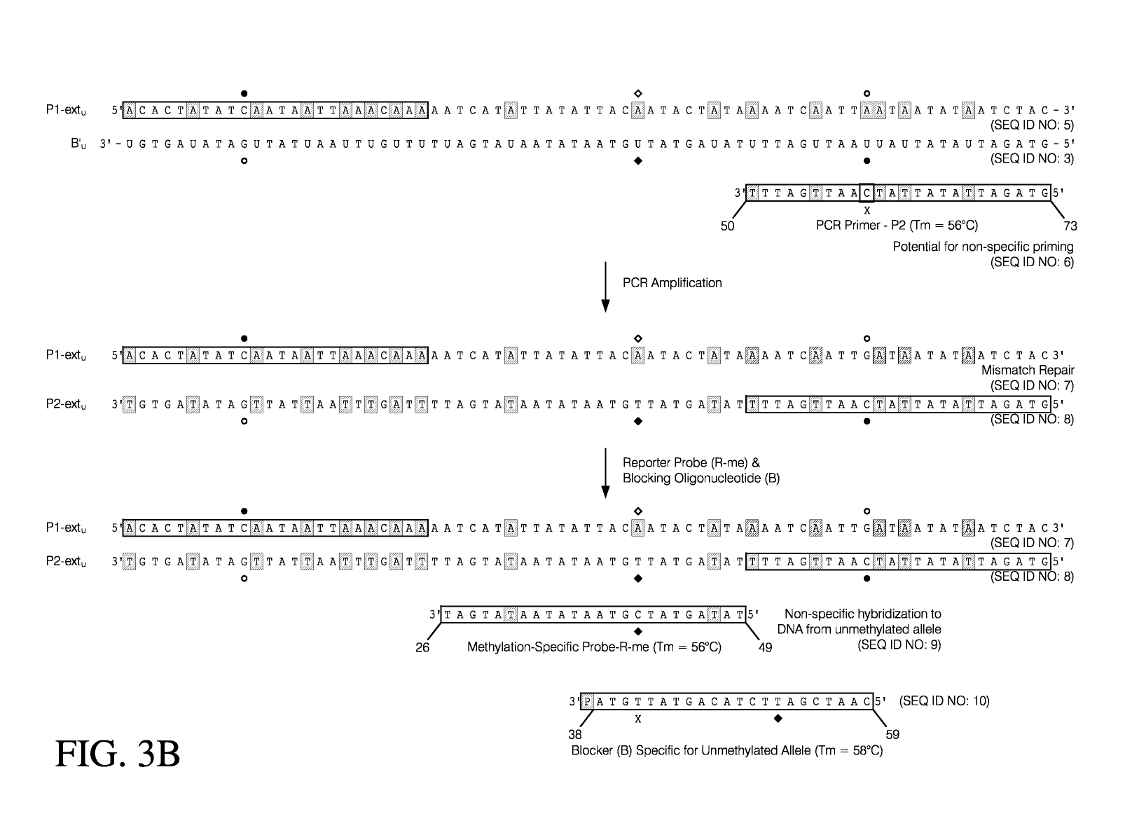

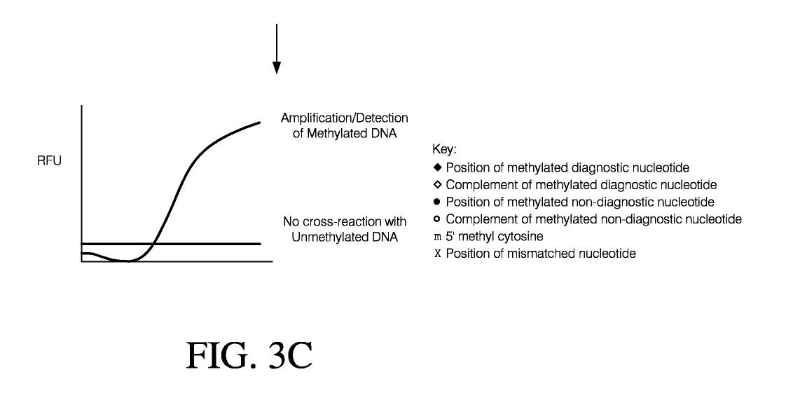

In some embodiments, the sample DNA is treated with an agent the selectively modifies unmethylated cytosine residues. By way of example only, in some embodiments, the sample nucleic acids are treated with sodium bisulphite, according to art-accepted methods. (See, e.g., Formmer, et al. (1992) Proc. Nat. Acad. Sci. USA 89:1827-1831). Treatment with sodium bisulphite sulphonates unmethylated cytosines, but not methylated cytosines. Following sulphonation, the sample is subjected to conditions (e.g., alkaline conditions, or any other appropriate conditions), that deaminate the sulphonated DNA to yield a uracil-bisulphite derivative that is in turn converted to uracil by alkaline desulphonation. Selective conversion of the unmethylated cytosine residues on both strains (i.e., the first strand and the second strand) generates novel sequences, referred to as “modified target DNA,” for convenience, as illustrated in FIG. 3. The modified sample nucleic acids are then subjected to an amplification (and/or detection) reaction, as discussed below.

In some embodiments, provided herein are methods to detect, or enhance the specificity of detection of rare methylation events, e.g., by performing a methylation-specific amplification reaction (e.g., methylation specific PCR). Modified sample nucleic acids are contacted with a forward and a reverse amplification primer that specifically hybridize to opposite strands of the modified sample nucleic acids, i.e., the forward primer hybridizes to the first strand of the modified nucleic acids (e.g., modified sample nucleic acids, or modified target DNA) and the reverse primer hybridizes to the second strand of the modified nucleic acids (e.g., modified sample nucleic acids, or modified target DNA), and amplify the region between the two primers under amplification conditions.

Referring to Fig. 3, the forward primer (P1), comprises a sequence that is complementary to (specifically hybridizes to) modified target DNA B, the target nucleotide sequence of the second strand following cytosine modification, i.e., the unique sequence generated by specific modification of unmethylated cytosine residues as discussed above. The forward primer thus contains one or more adenine residues that are located in the primer to hybridize to uracil residues present in the modified sample nucleic acids (e.g., modified sample nucleic acids, or modified target DNA). Accordingly, in some embodiments, the forward primer comprises one or more adenine residues that will base-pair with uracil residues in the second strand template sequence (converted from unmethylated cytosine residues in the second strand original sample sequence), i.e., modified target DNA B as shown in FIG. 3. In some embodiments, the one or more adenine residues that base-pair with uracil residues in the template sequence include an adenine residue located at the 3′ end of the forward primer P1, as shown in FIG. 3. As such, extension will occur when the original sample DNA prior to modification of the unmethylated cytosines (e.g., by bisulphite treatment), comprises an unmethylated cytosine residue at the same position (shown in FIG. 3). If the second strand of the template contains methylated cytosine residues, then treatment with bisulphite will not generate a novel sequence, and the adenosine residues in the methylation-specific primer will be mismatched with the methylated cytosines in the second strand of the template nucleic acids. As such, amplification will not occur when the second strand of the original sample nucleic acids (prior to modification) comprises a methylated cytosine residue at the same position (not shown). In some embodiments, the forward primer is fully complementary to a target sequence that comprises methylated cytosines and is also fully complementary to a target sequence that comprises unmethylated cytosines (see, e.g., EXAMPE 2, below). For example, in some embodiments, the forward primer hybridizes to a target sequence that does not include potentially methylated cytosine residues.

In some embodiments, the reverse primer (depicted as P2 in FIG. 3) is complementary to the unique first strand sequence generated by amplification from the forward primer following modification of the sample nucleic acids. The unique first strand sequence generated by amplification is depicted as P1-extᵤ in FIG. 3. Accordingly, in some embodiments, the reverse primer comprises one or more thymine residues, which correspond to the position of one or more uracil residues (converted from unmethylated cytosine residues in the second strand original sample sequence, i.e., modified target DNA B), and that base-pair with adenine residues present in the extension product from the forward primer (P1-extᵤ). In some embodiments, the one or more thymine residues corresponding to the position of one or more uracil residues (converted from unmethylated cytosine residues in the second strand original sample sequence), is at the 3′ end of the reverse primer. As such, extension will occur when the second strand of the original sample DNA comprises an unmethylated cytosine residue at the same position (shown in FIG. 3), and will not occur when the second strand of the original sample DNA comprises a methylated cytosine residue at the same position (not shown). The extension product from P2 is depicted as P2-extᵤ in FIG. 3.

In some embodiments, the methods comprise contacting the treated sample (e.g., a sample that has been treated to selectively modify cytosine residues) with methylation-specific forward and reverse primers as described herein, under amplification conditions, as described below. In some embodiments, the methods include contacting the treated sample with a methylation-specific probe (e.g., by including the methylation-specific probe in the reaction mixture prior to amplification, or by contacting the sample with the methylation-specific probe post-amplification). Methylation-specific probes can include sequences that are complementary to and thus hybridize to the unique amplicons produced by successful extension from the forward and reverse methylation-specific primers, as described above. In some embodiments, the methylation specific probe comprises one or more cytosine residues that correspond to the position of a methylated cytosine residue present in the sample nucleic acids (e.g., and that are thus present as cytosine residues on the P2-extᵤ strand, or second strand of the amplified, modified target sequences). As shown in FIG. 3, the methylated cytosine residues are not converted to uracil by bisulphite treatment, and thus the first and second strands of the amplicons produced by P1 and P2 (P1-extᵤ and P2-extᵤ, respectively, in FIG. 3) contain a guanine-cytosine base pair. In some embodiments, the methylation specific probe (shown as Rₘₑ in FIG. 3) also contains one or more thymine residues that correspond to the position of an unmethylated cytosine residue in the sample nucleic acids (and thus, a uracil residue in the modified sample nucleic acids, modified target DNA B). In some embodiments, the methylation-specific probe contains a detectable label or detectable moiety, as discussed in further detail below.

In some embodiments, the amplification reaction mixture also includes a “modulator oligonucleotide” or “blocking oligonucleotide.” In some embodiments, modulator oligonucleotides or blocker oligonucleotides are used selectively suppress non-specific hybridization of the methylation-specific amplification primers and/or methylation-specific reporter probes. Accordingly, modulator oligonucleotides or blocking probes can be used to overcome the potential for false positive results owing to the presence of mixed populations of methylated and unmethylated target nucleic acid sequences, as may be encountered in clinical samples. As shown in FIG. 3, in some embodiments, a blocking probe is used to enhance the specificity of methylation-specific amplification. For example, in some embodiments, the blocking probe (shown as “B” in FIG. 3) that competes with both primer P2 and the reporter probe Rₘₑ for hybridization with the amplified target. The sequence of the modulator oligonucleotide or blocking oligonucleotide B is designed such that it preferentially hybridizes, in this case, to amplification product derived from unmethylated DNA target strand Aᵤ. The Tₘ of the modulator oligonucleotide or blocking oligonucleotide B is designed to be substantially similar to the Tₘ of the forward and reverse methylation-specific amplification primers (P1 and P2, and, reporter probe Rₘₑ). In some embodiments, the Tₘ of the blocking probe differs by less than 15° C., 14° C., 13° C., 12° C., 11° C., 10° C., 9° C., 8° C., 7° C., 6° C., 5° C., 4° C., 3° C., 2° C., or 1° C., or less, from the methylation-specific amplification primers and/or reporter probe. As such, in some embodiments, the reactions are optimized to allow discrimination between methylated an unmethylated DNA forms, e.g., by balancing concentration and the conditions of hybridization (in particular temperature and salt concentration, as well as other factors known in the art). In general, the higher the Tₘ of the blocking probe relative to that of the primer and/or reporter probe with which it competes, the lower the concentration of probe required to suppress non-specific amplification and/or detection of target nucleic acids. As discussed in further detail below, the blocker oligonucleotides are designed such that they cannot be extended from their 3′ ends.

### Amplification Primers

Amplification primers useful in the embodiments disclosed herein are preferably between 10 and 45 nucleotides in length. For example, the primers can be at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or more nucleotides in length. Primers can be provided in any suitable form, included bound to a solid support, liquid, and lyophilized, for example. In some embodiments, the primers and/or probes include oligonucleotides that hybridize to a reference nucleic acid sequence over the entire length of the oligonucleotide sequence. Such sequences can be referred to as “fully complementary” with respect to each other. Where an oligonucleotide is referred to as “substantially complementary” with respect to a nucleic acid sequence herein, the two sequences can be fully complementary, or they may form mismatches upon hybridization, but retain the ability to hybridize under stringent conditions or standard PCR conditions as discussed below. As used herein, the term “standard PCR conditions” include, for example, any of the PCR conditions disclosed herein, or known in the art, as described in, for example, PCR 1: A Practical Approach, M. J. McPherson, P. Quirke, and G. R. Taylor, Ed., (c) 2001, Oxford University Press, Oxford, England, and PCR Protocols: Current Methods and Applications, B. White, Ed., (c) 1993, Humana Press, Totowa, N.J. The amplification primers can be substantially complementary to their annealing region, comprising the specific variant target sequence(s) or the wild type target sequence(s). Accordingly, substantially complementary sequences can refer to sequences ranging in percent identity from 100, 99, 98, 97, 96, 95, 94, 93, 92, 91, 90, 89, 85, 80, 75 or less, or any number in between, compared to the reference sequence. Conditions for enhancing the stringency of amplification reactions and suitable in the embodiments disclosed herein, are well-known to those in the art. A discussion of PCR conditions, and stringency of PCR, can be found, for example in Roux, K. “Optimization and Troubleshooting in PCR,” in PCR PRIMER: A LABORATORY MANUAL, Diffenbach, Ed. © 1995, Cold Spring Harbor Laboratory Press, Cold Spring Harbor, N.Y.; and Datta, et al. (2003) Nucl. Acids Res. 31(19):5590-5597.

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml ), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and %SDS) less stringent that those described above. An example of moderately stringent conditions is overnight incubation at 37° C. in a solution comprising: 20% formamide, 5×SSC (150 mM NaCl 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as oligonucleotide length and the like.

In some embodiments, primer pairs comprising a forward and reverse primer are used in the amplification methods described herein, e.g., to produce target amplicons. In some embodiments, the Tₘ of the forward and reverse primers are substantially similar, e.g., differ by less than 15° C., 14° C., 13° C., 12° C., 11° C., 10° C., 9° C., 8° C., 7° C., 6° C., 5° C., 4° C., 3° C., 2° C., or 1° C., or less.

### Blocker Oligonucleotides

In an amplification reaction wherein reagents such as polymerase and dNTPs are limiting, when a sample comprises a large excess of wild-type target sequences compared to variant or mutant target sequences or alleles, (e.g., 10 fold, 100 fold, 1000 fold or more excess of wild-type target sequence compared to variant or mutant sequence), the kinetics of the amplification reaction are driven such that the limiting reagents are consumed in the amplification of wild-type sequences, while amplification and/or detection of the rare variant, rare mutant, alleles is suppressed. In order to shift the equilibrium to favor amplification of the rare variant or mutant alleles, blocker oligonucleotides can be added to the reaction.

As used herein, the term “blocker oligonucleotide” refers to an oligonucleotide that binds to a strand of DNA within the target amplicon, and that is designed to preferentially bind to the wild-type allele sequence (e.g., the abundant allelic sequence, such as a wild-type allele sequence) compared to the target variant sequence (e.g., the rare allelic variant). The blocker oligonucleotide generally comprises a modification, or modifications, as discussed below, that prevent primer extension by a polymerase. Thus, a blocker oligonucleotide can tightly bind to a wild type allele in order to suppress amplification of the wild-type allele while amplification of the variant target allele sequence is allowed to occur. As explained above, blocker oligonucleotides can also be advantageously used in the methods described herein for the detection of methylation variants, e.g., in methylation specific amplification reactions as discussed above.

Blocker oligonucleotides as disclosed herein refer to oligonucleotides that are incapable of extension by a polymerase, for example, when hybridized to its complementary sequence in an amplification assay, e.g., PCR. Several different means of modifying oligonucleotides to render them incapable of extension by a polymerase are known and useful in the embodiments disclosed herein. By way of example, common examples of oligonucleotide modifications include, for example, 3′-OH modifications and dideoxy nucleotides. Numerous 3′-OH blocking materials are known and suitable, and include cordycepin (3′-deoxyadenosine) and other 3′-moieties such as those described in Josefen, M. et al. (2009) Mol. Cell Probes 23:201-223 McKinzie, P. et al. (2006) Mutagenesis , 21(6):391-397; Parson, B. et al. (2005) Methods Mol. Biol., 291:235-245; Parsons, B. et al. (1992) Nucl. Acids. Res., 25:20(10):2493-2496, and Morlan, J. et al. (2009) PLoS One 4(2):e4584, the disclosures of which relating to oligonucleotide modifications are hereby incorporated by reference. In some embodiments, the 3′-OH is blocked with a (3-amino-2-hydroxy)-propoxyphosphoryl. In some embodiments, the 3′-OH is blocked by introduction of a 3′-3′-A-5′ linkage such as those described in U.S. Pat. No. 5,660,989.

In some embodiments, the blocker oligonucleotide comprises a moiety that binds within the minor groove of double-stranded DNA at its 3′ end, which prevents polymerase extension. A variety of moieties that bind to the minor groove of DNA suitable for the blocker oligonucleotides disclosed herein are known in the art, and include, but are not limited to those described in U.S. Pat. No. 5,801,155, Wemmer, et al. (1997) Curr. Opin. Structural Biol. 7:355-361, Walker, et al. (1997) Biopolymers 44:323-334, Zimmer, et al. (1986) Molec. Biol. 47:31-112, and Reddy, B. et al. (1999) Pharmacol. Therap. 84:1-111. Methods for incorporating or attaching minor-groove binding moieties to oligonucleotides are well-known. For example, methods described in U.S. Pat. Nos. 5,512,677, 5,419,966, 5,696,251, 5,585,481, 5,492,610, 5,736,626, 5,801,155 and 6,727,356 are suitable for modifying oligonucleotides to generate a blocking oligonucleotide.

In some embodiments, the blocking oligonucleotides disclosed herein can include a minor-groove binding moiety located at the 5′ end, the 3′ end, or at a position within the oligonucleotide.

The skilled artisan will readily appreciate that the exemplary “blocking” modifications discussed above are provided by way of illustration only, and that any blocking modification known or discovered in the future can be used in the blocking oligonucleotides and methods disclosed herein.

In some embodiments, the blocker oligonucleotides comprise one or more modifications that increase the Tₘ of the oligonucleotide. For example, in some embodiments the blocker oligonucleotide can comprise one or more nucleosidic bases different from the naturally occurring bases (i.e., adenine, cytosine, thymine, guanine and uracil). In some embodiments, the modified bases effectively hybridize to nucleic acid units that contain naturally occurring bases. In some embodiments, the modified base(s) increase the difference in the Tₘ between matched and mismatched sequences, and/or decrease mismatched priming efficiency, thereby improving the specificity and sensitivity of the assay.

Non-limiting examples of modified bases useful in the embodiments disclosed herein include the general class of base analogues 7-deazapurines and their derivatives and pyrazolopyrimidines and their derivatives (described in PCT WO 90/14353; and U.S. application Ser. No. 09/054,630, the disclosures of each of which are incorporated herein by reference in regards to the base analogues). Examples of base analogues of this type include, for example, the guanine analogue 6-amino-1H-pyrazolo[3,4-d]pyrimidin-4(5H)-one (ppG), the adenine analogue 4-amino-1H-pyrazolo[3,4-d]pyrimidine (ppA), and the xanthine analogue 1H-pyrazolo[4,4-d]pyrimidin-4(5H)-6(7H)-dione (ppX). These base analogues, when present in an oligonucleotide of some embodiments of the methods and compositions disclosed herein, strengthen hybridization.

Additionally, in some embodiments, modified sugars or sugar analogues can be present in one or more of the nucleotide subunits of a blocker oligonucleotide. Sugar modifications useful in the embodiments disclosed herein include, but are not limited to, attachment of substituents to the 2′, 3′ and/or 4 ′ carbon atom of the sugar, different epimeric forms of the sugar, differences in the α or β-configuration of the glycosidic bond, and other anomeric changes. Sugar moieties useful in the embodiments disclosed herein include, but are not limited to, pentose, deoxypentose, hexose, deoxyhexose, ribose, deoxyribose, glucose, arabinose, pentofuranose, xylose, lyxose, and cyclopentyl.

In some embodiments the blocker oligonucleotide can contain one or more locked nucleic acid (LNA)-type modifications. LNA modifications useful in the embodiments disclosed herein can involve alterations to the pentose sugar of ribo- and deoxyribonucleotides that constrains, or “locks,” the sugar in the N-type conformation seen in A-form DNA. In some embodiments, this lock can be achieved via a 2′-O, 4′-C methylene linkage in 1,2:5,6-di-O-isopropylene-.alpha.-D-allofuranose. In other embodiments, this alteration then serves as the foundation for synthesizing locked nucleotide phosphoramidite monomers. (See, for example, Wengel J., Ace. Chem. Res., 32:301-310 (1998), U.S. Pat. No. 7,060,809; Obika, et al., Tetrahedron Lett 39: 5401-5405 (1998); Singh, et al., Chem Commun 4:455-456 (1998); Koshkin, et al., Tetrahedron 54: 3607-3630 (1998), the disclosures of each of which are incorporated herein by reference

In some embodiments, modified bases useful in the embodiments disclosed herein include 8-Aza-7-deaza-dA (ppA), 8-Aza-7-deaza-dG (ppG), 2′-Deoxypseudoisocytidine (iso dC), 5-fluoro-2′-deoxyuridine (fdU), locked nucleic acid (LNA), or 2′-O, 4′-C-ethylene bridged nucleic acid (ENA) bases. Other examples of modified bases that can be used in the embodiments disclosed herein are described in U.S. Pat. No. 7,517,978 (the disclosure of which is incorporated herein by reference).

Many modified bases, including for example, LNA, ppA, ppG, 5-Fluoro-dU (fdU), are commercially available and can be used in oligonucleotide synthesis methods well known in the art. In some embodiments, synthesis of modified primers and probes can be carried out using standard chemical means also well known in the art. For example, in certain embodiments, the modified moiety or base can be introduced by use of a (a) modified nucleoside as a DNA synthesis support, (b) modified nucleoside as a phosphoramidite, (c) reagent during DNA synthesis (e.g., benzylamine treatment of a convertible amidite when incorporated into a DNA sequence), or (d) by post-synthetic modification according to art-accepted techniques.

In some embodiments, the primers or probes are synthesized so that the modified bases are positioned at the 3′ end of the blocker oligonucleotide. In some embodiments, the modified base are located between, 1-6 nucleotides, e.g., 2, 3, 4 or 5 nucleotides away from the 3′-end of the blocker oligonucleotide.

Modified internucleotide linkages can also be present in oligonucleotides, e.g., the blocker oligonucleotides in the embodiments disclosed herein. Modified linkages useful in the embodiments disclosed herein include, but are not limited to, peptide, phosphate, phosphodiester, phosphodiester, alkylphosphate, alkanephosphonate, thiophosphate, phosphorothioate, phosphorodithioate, methylphosphonate, phosphoramidate, substituted phosphoramidate and the like. Several further modifications of bases, sugars and/or internucleotide linkages, that are compatible with their use in oligonucleotides serving as probes and/or primers, will be apparent to those of skill in the art.

In some embodiments, the blocker oligonucleotide binds to a sequence which overlaps with the annealing region of the forward or reverse amplification primer. For example, in some embodiments, the blocker oligonucleotide and the forward or reverse primer are identical across 5, 6, 7, 8, 9, 10, 11, 12, 13, 14, 15, 16, 17, 18, 19, 20, 21, 22, 23, 24, 25, or more consecutive nucleotides. In some embodiments, the overlap in sequence identity between the blocker oligonucleotide and the forward or reverse amplification primer exists over 10%, 15%, 20%, 25%, 30%, 35%, 40%, 45%, 50%, 55%, 60%, 65%, 70%, 75%, 80%, 85%, or more, or any percentage in between, of the length of the blocker oligonucleotide and/or amplification primer. In some embodiments, the amplification primer comprises one or more nucleotides, e.g., 1, 2, 3, 4, 5, 6, 7, 8, 9, 10, 11, 12, 13, 14, 15, 16, 17, 18, 19, 20, 21, 22, 23, 24, 25 or more, on its 5′ end that are not identical to the blocker oligonucleotide (but that are complementary or substantially complementary to the reference sequence). In some embodiments, the blocker oligonucleotide comprises one or more nucleotides, e.g., 1, 2, 3, 4, 5, 6, 7, 8, 9, 10, 11, 12, 13, 14, 15, 16, 17, 18, 19, 20, 21, 22, 23, 24, 25 or more, on its 3′ end that are not identical to the amplification primer (but that are complementary or substantially complementary to the reference sequence).

As shown in FIGS. 1 and 2, the blocker oligonucleotide preferentially binds to the wild-type target sequence compared to the mutant or variant target sequence. Also shown in FIGS. 1 and 2 is the overlap between the amplification primer (i.e., primer 1 as shown) and the blocker oligonucleotide. As shown in FIGS. 1 and 2, binding of the blocker oligonucleotide to the wild type allele target sequence prevents binding and extension of the amplification primer, thereby suppressing amplification of the wild-type sequence. In contrast to the wild-type allele sequence, the amplification primer will preferentially bind to the mutant allele sequence, over the blocking oligonucleotide. Thus, the amplification is not blocked and the amplification of the mutant target allele sequence proceeds unimpeded. By this means, the present method advantageously allows for simultaneous and preferential amplification of one or more variant or mutant target allele sequences.

### Reporter Probes

To detect the presence and/or amount of variant target sequence(s) e.g., rare variant or mutant template nucleic acids in the sample, the sample is contacted with one or more allele-specific reporter probes. In some embodiments, the methods disclosed herein provide for the detection of more than one variant or mutant allele sequence in a sample. Accordingly, in some embodiments, a sample can be contacted with 1, 2, 3, 4, 5, 6, 7, 8 or more, reporter probes. Each reporter probe preferentially binds to a cognate allelic variant compared to the wild type allelic sequence. As discussed above, in some embodiments, reporter probes can be advantageously used to detect methylation variants, e.g., in methylation-specific amplification as discussed above.

The reporter probes can comprise a detectable moiety. In some embodiments, the probe can include a detectable label. Labels of interest include directly detectable and indirectly detectable radioactive or non-radioactive labels such as fluorescent dyes and the like. Directly detectable labels refer to detectable moieties that provide a directly detectable signal without interaction with one or more additional chemical agents. Indirectly detectable labels are those labels which interact with one or more additional members to provide a detectable signal. In this latter embodiment, the label is a member of a signal producing system that includes two or more chemical agents that work together to provide the detectable signal. Examples of indirectly detectable labels include biotin or digoxigenin, which can be detected by a suitable antibody coupled to a fluorochrome or enzyme, such as alkaline phosphatase.

In some embodiments, the label is a directly detectable label. Directly detectable labels of particular interest include fluorescent labels. Fluorescent labels suitable in the detector probes of the embodiments disclosed herein include fluorophore moieties. Specific fluorescent dyes of interest include: xanthene dyes, e.g., fluorescein and rhodamine dyes, such as fluorescein isothiocyanate (FITC), 2-[ethylamino)-3-(ethylimino)-2-7-dimethyl-3H-xanthen-9-yl]benzoic acid ethyl ester monohydrochloride (R6G)(emits a response radiation in the wavelength that ranges from about 500 to 560 nm), 1,1,3,3,3 ′, 3′-Hexamethylindodicarbocyanine iodide (HIDC) (emits a response radiation in the wavelength that ranged from about 600 to 660 nm), 6-carboxyfluorescein (commonly known by the abbreviations FAM and F), 6-carboxy-2′,4′,7′,4,7-hexachlorofluorescein (HEX), 6-carboxy-4′, 5′-dichloro-2′,7′-dimethoxyfluorescein (JOE or J), N,N,N′,N′-tetramethyl-6-carboxyrhodamine (TAMRA or T), 6-carboxy-X-rhodamine (ROX or R), 5-carboxyrhodamine 6G (R6G5 or G5), 6-carboxyrhodamine-6G (R6G6 or G6), and rhodamine 110; cyanine dyes, e.g. Cy3, Cy5 and Cy7 dyes; coumarins, e.g., umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3 (emits a response radiation in the wavelength that ranges from about 540 to 580 nm), Cy5 (emits a response radiation in the wavelength that ranges from about 640 to 680 nm), etc; BODIPY dyes and quinoline dyes. Specific fluorophores of interest include: Pyrene, Coumarin, Diethylaminocoumarin, FAM, Fluorescein Chlorotriazinyl, Fluorescein, R110, Eosin, JOE, R6G, HIDC, Tetramethylrhodamine, TAMRA, Lissamine, ROX, Napthofluorescein, Texas Red, Napthofluorescein, Cy3, and Cy5, and the like. In preferred embodiments, the reporter probe can be a molecular beacon probe, a TAQMAN™ probe, or a SCORPION™ probe.

In some embodiments, the reporter probe(s) have a Tₘ that is higher than the Tₘ of the forward and reverse amplification primers used in the methods disclosed herein. For example, in some embodiments, the probes, e.g., molecular beacon probes or the like, have a Tₘ that is greater than 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., or 25° C., or more than either amplification primer used to generate an amplicon to which the oligonucleotide probe hybridizes. For example, a molecular beacon probe can have a Tₘ that is at least 5-10° C. higher than either amplification primer pair used to generate the amplicon to which the molecular beacon hybridizes. In some embodiments, the reporter probe(s) have a Tₘ that is the same or lower than the forward and reverse amplification primers disclosed herein.

As used herein, the term “Tm” and “melting temperature” are interchangeable terms which refer to the temperature at which 50% of a population of double stranded polynucleotide molecules become dissociated into single strands. The Tm of particular nucleic acids, e.g., primers, or oligonucleotide probes, or the like can be readily calculated by the following equation: Tm=69.3+0.41×(G+C)%-650/L, wherein L refers to the length of the nucleic acid. The Tm of a hybrid polynucleotide may also be estimated using a formula adopted from hybridization assays in 1 M salt, and is commonly used for calculating the Tm for PCR primers: [(number of A+T)×2° C.+(number of G+C)×4° C ], see, for example, Newton et al. (1997) PCR (2nd ed; Springer-Verlag, New York). Other more sophisticated computations exist in the art, which take structural as well as sequence characteristics into account for the calculation of Tm. A calculated Tm is merely an estimate; the optimum temperature is commonly determined empirically.

In some embodiments, the reporter probe can comprise an oligonucleotide that is shorter in length than the forward or reverse amplification primer. For example, in some embodiments, the reporter probe(s) is 1, 2, 3, 4, 5, 6, 7, 8, 9, 10, or more nucleotides shorter than either the forward or reverse amplification primer.

In some embodiments, the reporter probe(s) bind to an overlapping sequence, as the blocker oligonucleotide. For example, in some embodiments, the reporter probe(s) and the blocker oligonucleotide are identical across 5, 6, 7, 8, 9, 10, 11, 12, 13, 14, 15, 16, 17, 18, 19, 20, 21, 22, 23, 24, 25, or more consecutive nucleotides. In some embodiments, the overlap in sequence identity between the reporter probe(s) and the blocker oligonucleotide exists over 10%, 15%, 20%, 25%, 30%, 35%, 40%, 45%, 50%, 55%, 60%, 65%, 70%, 75%, 80%, 85%, or more, or any percentage in between, of the length of the blocker oligonucleotide and/or reporter probe(s). In some embodiments, the blocker oligonucleotide comprises one or more nucleotides, e.g., 1, 2, 3, 4, 5, 6, 7, 8, 9, 10, 11, 12, 13, 14, 15, 16, 17, 18, 19, 20, 21, 22, 23, 24, 25 or more, on its 5′ end that are not identical to the reporter probe (but that are complementary or substantially complementary to the reference sequence). In some embodiments, the reporter probe comprises one or more nucleotides, e.g., 1, 2, 3, 4, 5, 6, 7, 8, 9, 10, 11, 12, 13, 14, 15, 16, 17, 18, 19, 20, 21, 22, 23, 24, 25 or more, on its 3′ end that are not identical to the blocker probe (but that are complementary or substantially complementary to the reference sequence).

As shown in FIGS. 1 and 2, the reporter probe(s) is allele-specific. That is, the reporter probe is complementary to the variant or mutant allele sequence(s) being assayed, and non-complementary to the wild-type allele sequence. As shown in FIGS. 1 and 2, binding of the detector probe to the mutant or variant target allele sequence does not block or impede amplification by the amplification primers. Binding of the reporter probe to the mutant allele sequence (e.g., within sample template sequence or amplicon sequences) produces a detectable signal. As shown in FIG. 2, in some embodiments, reaction mixtures can contain more than one detector probe, wherein each detector probe is specific for a different variant or mutant target allele sequence, and wherein each detector probe comprises a different detectable moiety. Accordingly, detection and identification of different mutant alleles in a single sample/reaction mixture is possible.

In addition to the sample, amplification primers, blocker oligonucleotide, and reporter probe(s), the reaction mixture includes a polymerase. The skilled artisan will appreciate that many polymerases known to those in the art are suitable for the methods described herein. For example, thermostable polymerases (including commercially available polymerases) obtained from Thermus aquaticus, Thermus thermophilus, Thermococcus litoralis, Pyrococcus furiosus, Pyrococcus woosii and other species of the Pyrococcus genus, Bacillus stearothermophilus, Sulfolobus acidocaldarius, Thermoplasma acidophilum, Thermus flavus, Thermus ruber, Thermus brockianus, Thermotoga neapolitana, Thermotoga maritima and other species of the Thermotoga genus, and Methanobacterium thermoautotrophicum, and mutants of each of these species are useful in the embodiments disclosed herein. Preferable thermostable polymerases can include, but are not limited to, Taq DNA polymerase, Th DNA polymerase, Tma DNA polymerase, or mutants, derivatives or fragments thereof.

Usually the reaction mixture will further comprise four different types of dNTPs corresponding to the four naturally occurring nucleoside bases, i.e., dATP, dTTP, dCTP, and dGTP. In the methods of the invention, each dNTP will typically be present in an amount ranging from about 10 to 5000 μM, usually from about 20 to 1000 μM, about 100 to 800 μM, or about 300 to 600 μM.

The reaction mixture can further include an aqueous buffer medium that includes a source of monovalent ions, a source of divalent cations, and a buffering agent. Any convenient source of monovalent ions, such as potassium chloride, potassium acetate, ammonium acetate, potassium glutamate, ammonium chloride, ammonium sulfate, and the like may be employed. The divalent cation may be magnesium, manganese, zinc, and the like, where the cation will typically be magnesium. Any convenient source of magnesium cation may be employed, including magnesium chloride, magnesium acetate, and the like. The amount of magnesium present in the buffer may range from 0.5 to 10 mM, and can range from about 1 to about 6 mM, or about 3 to about 5 mM. Representative buffering agents or salts that may be present in the buffer include Tris, Tricine, HEPES, MOPS, and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, for example, about pH 6.0, 6.5, 7.0, 7.5, 8.0, 8.5, 9.0, or 9.5. Other agents that may be present in the buffer medium include chelating agents, such as EDTA, EGTA, and the like. In some embodiments, the reaction mixture can include BSA, or the like. In addition, in some embodiments, the reactions can include a cryoprotectant, such as trehalose, particularly when the reagents are provided as a master mix, which can be stored over time.

In preparing a reaction mixture, the various constituent components may be combined in any convenient order. For example, the buffer may be combined with primer, polymerase, and then template nucleic acid, or all of the various constituent components may be combined at the same time to produce the reaction mixture.

Alternatively, commercially available premixed reagents can be utilized in the methods disclosed herein, according to the manufacturer's instructions, or modified to improve reaction conditions (e.g., modification of buffer concentration, cation concentration, or dNTP concentration, as necessary), including, for example, TAQMAN® Universal PCR Master Mix (Applied Biosystems), OMNIMIX® or SMARTMIX® (Cepheid), IQ&#8482; Supermix (Bio-Rad Laboratories), LIGHTCYCLER® FastStart (Roche Applied Science, Indianapolis, Ind.), or BRILLIANT® QPCR Master Mix (Stratagene, La Jolla, Calif.).

The reaction mixture can then be subjected to amplification, or primer extension conditions. For example, in some embodiments, the reaction mixture is subjected to thermal cycling or isothermal amplification. Thermal cycling conditions can vary in time as well as in temperature for each of the different steps, depending on the thermal cycler used as well as other variables that could modify the amplification's performance. In some embodiments, a 2-step protocol is performed, in which the protocol combines the annealing and elongation steps at a common temperature, optimal for both the annealing of the primers and probes as well as for the extension step. In some embodiments, a 3-step protocol is performed, in which a denaturation step, an annealing step, and an elongation step are performed.

In some embodiments, the compositions disclosed herein can be used in connection with devices for real-time amplification reactions, e.g., the BD MAX® (Becton Dickinson and Co., Franklin Lakes, N.J.), the VIPER® (Becton Dickinson and Co., Franklin Lakes, N.J.), the VIPER LT® (Becton Dickinson and Co., Franklin Lakes, N.J.), the SMARTCYLCER® (Cepheid, Sunnyvale, Calif.), ABI PRISM 7700® (Applied Biosystems, Foster City, Calif.), ROTOR-GENE™ (Corbett Research, Sydney, Australia), LIGHTCYCLER® (Roche Diagnostics Corp, Indianapolis, Ind.), ICYCLER® (BioRad Laboratories, Hercules, Calif.), IMX4000® (Stratagene, La Jolla, Calif.), CFX96™ Real-Time PCR System (Bio-Rad Laboratories Inc.), and the like.

In some embodiments, the compositions disclosed herein can be used in methods comprising isothermal amplification of nucleic acids. Isothermal amplification conditions can vary in time as well as temperature, depending on variables such as the method, enzyme, template, and primer or primers used. Examples of amplification methods that can be performed under isothermal conditions include, but are not limited to, some versions of LAMP, SDA, and the like.

Isothermal amplification can include an optional denaturation step, followed by an isothermal incubation in which nucleic acid is amplified. In some embodiments, an isothermal incubation is performed without an initial denaturing step. In some embodiments, the isothermal incubation is performed at least about 25° C., for example about 25° C., 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75° C., including ranges between any of the listed values. In some embodiments, the isothermal incubation is performed at about 37° C. In some embodiments, the isothermal incubation is performed at about 64° C. In some embodiments, the isothermal incubation is performed for 180 minutes or less, for example about 180, 165, 150, 135, 120, 105, 90, 75, 60, 45, 30, or 15 minutes, including ranges between any two of the listed values.

In some embodiments, the accumulation amplicons of the target sequences, i.e., the variant or mutant target allele sequence(s) are monitored in real-time. Methods for monitoring and assaying amplification reactions in real-time are widely known, and the skilled artisan will appreciate that any of the art-accepted techniques of real-time amplification are suitable for use in the embodiments disclosed herein. Exemplary descriptions of real-time amplification useful in the embodiments disclosed herein can be found, for example, in U.S. Pat. No. 6,783,984; U.S. Pat. No. 6,303,305, and the like. As used herein, the term “Ct” or “Ct value” refers to threshold cycle and signifies the cycle (or fractional cycle) of an amplification assay in which signal from a reporter that is indicative of amplicon generation (e.g., fluorescence), first become detectable above a background level. In some embodiments, the threshold cycle or “Ct” is the cycle number at which nucleic acid amplification becomes exponential. In some embodiments, e.g., in embodiments wherein amplification proceeds via isothermal amplification, threshold time values are used to signify the time in an amplification assay in which signal from a reporter that is indicative of amplicon generation (e.g., fluorescence), first becomes detectable above a background level. In some embodiments, the threshold time value is the time at which nucleic acid amplification becomes exponential.

As used herein, the term “delta Ct” or “ACt” refers to the difference in the numerical cycle number at which the signal passes a fixed threshold between two different samples or reactions. In some embodiments ΔCt refers to the difference in numerical cycle number at which exponential amplification is reached between two different samples or reactions. The ΔCt can be used to identify the specificity between a matched reporter probe to the corresponding target nucleic acid sequence and a mismatched reporter probe to the same corresponding sequence.

Various methods to calculate Ct values and threshold time values are known in the art and are useful in the embodiments disclosed herein. By way of example only, methods described in U.S. Patent Nos. 6,783,984, 6,303,305, and the like can be used in calculating Ct values and threshold time values in the methods disclosed herein. Accordingly, in some embodiments, the methods include the step of determining the Ct value or threshold time value, for each target allele sequence of interest (e.g., mutant or target allele sequences).

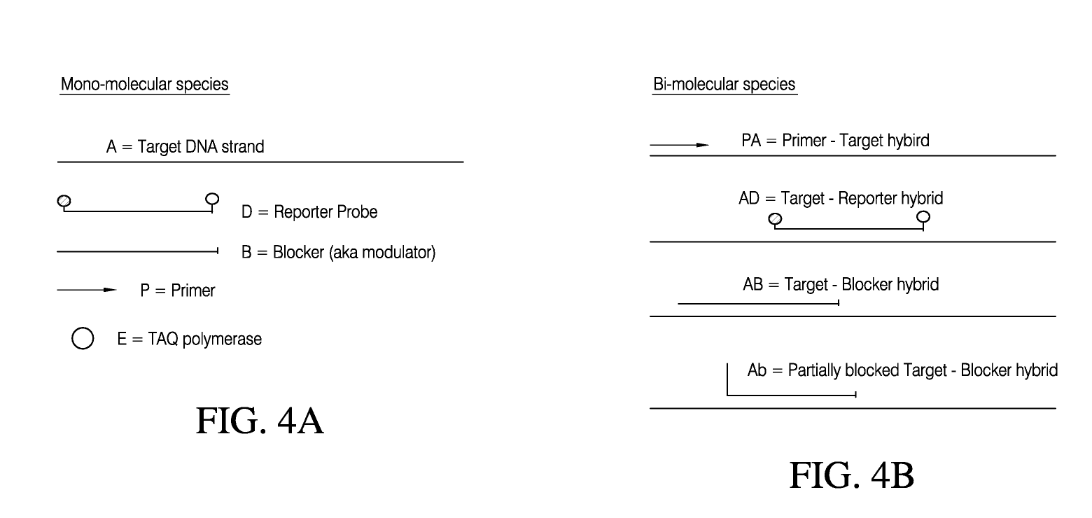

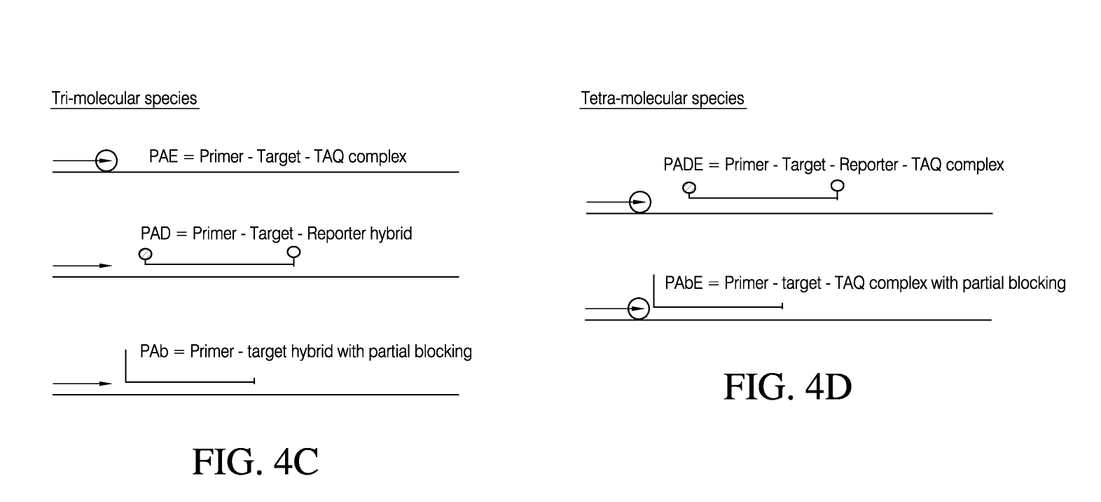

The present embodiments are based, in part, upon the discovery that using a combination of amplification primers, oligonucleotide blockers, and allele-specific detector probes, one can render amplification of rare allele sequences thermodynamically more favorable, thereby enabling their detection in samples that contain predominantly wild-type or other variant allele sequences. FIGS. 4-7 illustrate the concepts described herein, including the thermodynamic consideration used in practicing the embodiments disclosed herein.

FIG. 4 depicts the molecular species present in a reaction mixture that is subjected to primer extension or amplification conditions. “A” represents the “analyte” or target region of interest that comprises either the wild-type or variant or mutant allele sequence. As shown in FIG. 4A, the molecular species in the reaction mixture include the analyte, the reporter probe (“D”), the blocker oligonucleotide (“B”), the amplification primer(s) (“P”), and the polymerase (“E”). FIG. 4B shows bi-molecular species, including amplification primer bound to its cognate sequence on the analyte (“PA”), reporter probe bound to its cognate sequence on the analyte (“DA”), blocker oligonucleotide bound to its cognate sequence on the analyte (wild-type target allele sequence) (“BA”), and blocker oligonucleotide that is partially bound to the analyte (variant or mutant target allele sequence) (“Ab”). FIG. 4C depicts tri-molecular species, such as (1) complexes between the amplification primer, its cognate analyte, and polymerase (“PAE”); (2) complexes between the amplification primer, its cognate analyte, and a reporter probe (“PAD”); and (3) complexes between the amplification primer, its cognate analyte and an oligonucleotide blocker (“PAb”). FIG. 4D depicts possible tetra-molecular species, including (1) complexes between an amplification primer, its cognate analyte sequence, reporter probe, and polymerase (“PADE”); and (2) complexes between an amplification primer, its cognate analyte, a blocker oligonucleotide, and polymerase (“PAbE”). The PAb and PAbE species represent the case in which nucleotide at and near the 5′ end of the blocker are unhybridized to the analyte, but the remaining nucleotides of the blocker are hybridized to the analyte. In all cases, primers, probes, blockers may hybridize with wild-type or variant DNA; however the perfectly matched hybrids (e.g. blocker with wild-type DNA) will be thermodynamically more stable than hybrids containing mismatches (e.g. blocker with variant DNA).

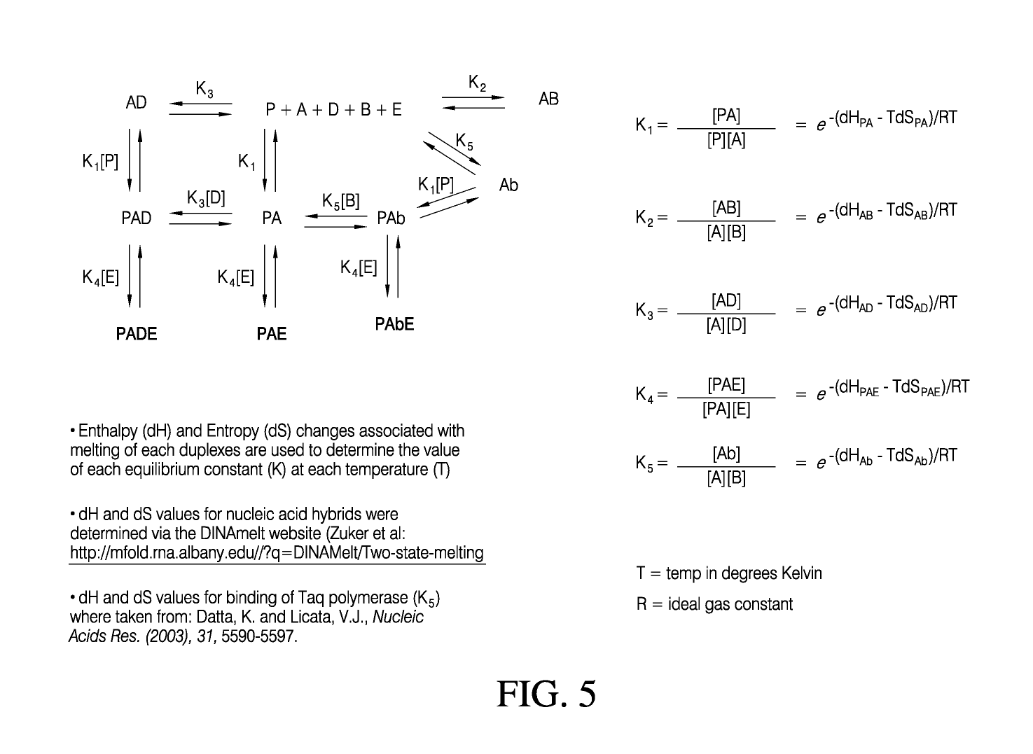

The molecular complexes shown in FIGS. 4A-4D exist in a multi-state equilibrium, as shown in FIG. 5. The association between each of the mono-molecular species is described by an equilibrium constant, K. The embodiments disclosed herein area based, in part, upon the discovery that equilibrium constants for the various molecular species shown in FIG. 4 can be advantageously used to model reaction conditions to maximize amplification of rare variant or mutant allele sequences compared in samples comprising an excess of copies (e.g., 5×, 10×, 20×, 30×, 40×, 50×, 100×, 500×, 750×, 1000×, or greater) of wild-type allele sequence compared to variant or mutant allele sequence, while minimizing detrimental effects on amplification efficiency. In accordance with the methods disclosed herein, the equilibrium constants for the complexes depicted in FIG. 4 can be estimated using enthalpy (dH) and entropy (dS) changes associated with melting of each of the duplexes, at each temperature. dH and dS values for each hybrid can be estimated or calculated using any art-accepted methods. By way of example, dH and dS can be calculated using publicly available algorithms, such as those available on the world wide web site hypertext transfer protocol://mfold.ma.albany.edu//?q=DINAMelt/Two-state-melting. The skilled artisan will appreciate that many known algorithms for calculation of dH and dS can be used in the methods disclosed herein. FIG. 5 shows the calculation of individual equilibrium constants according to the methods disclosed herein.

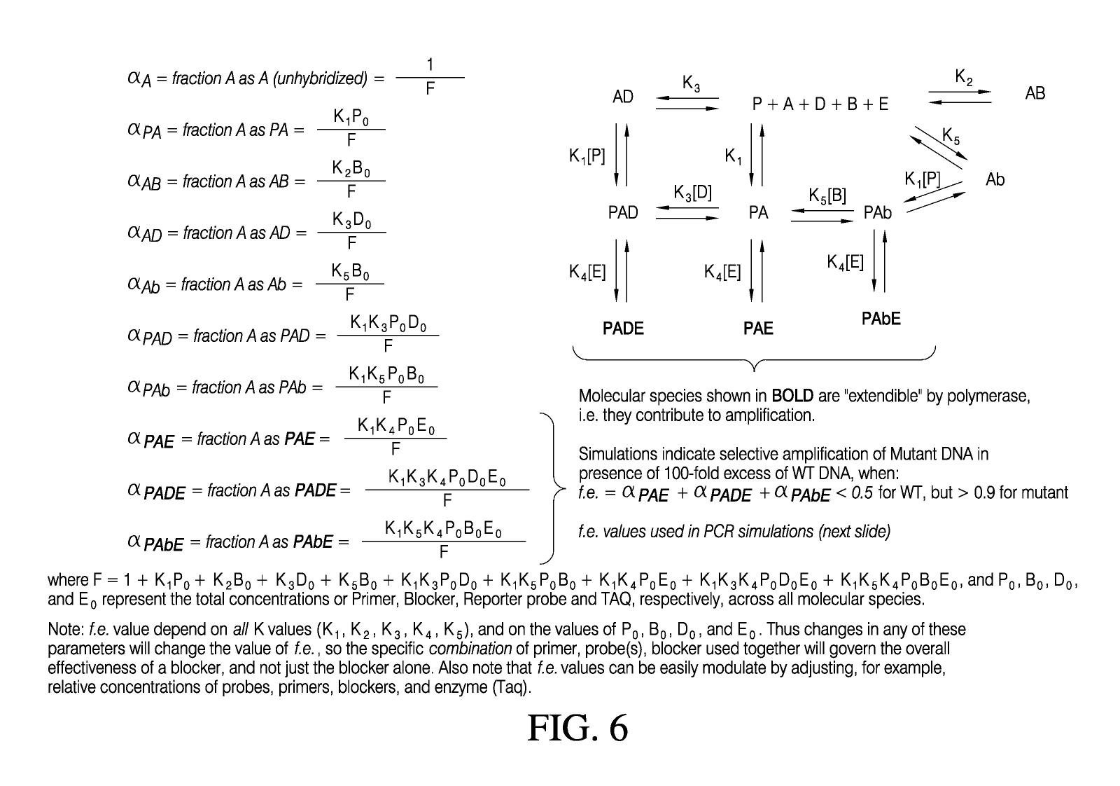

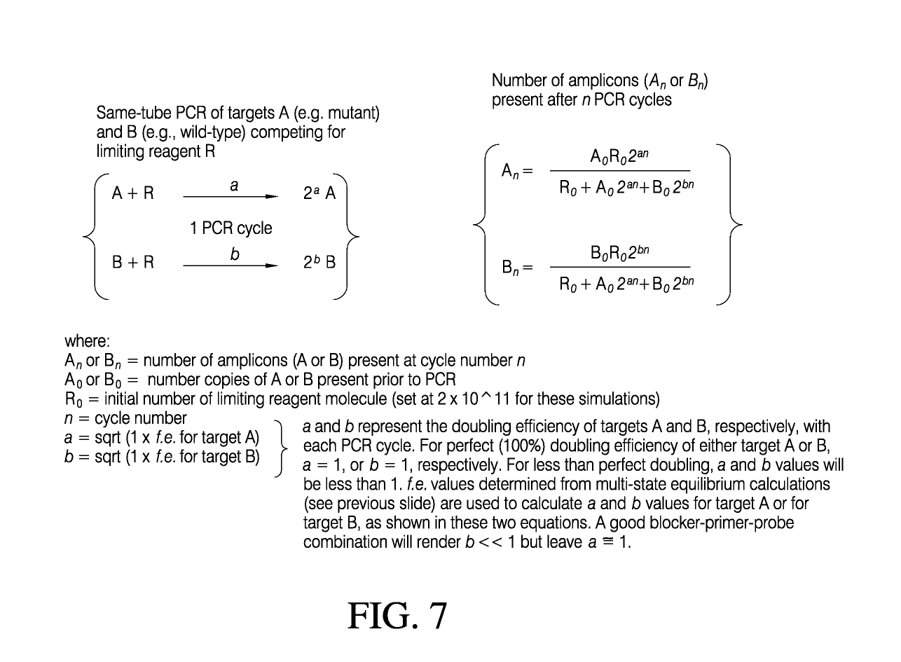

The present inventors discovered that equilibrium constants can be used to estimate the fraction of analyte “A” bound to blocker oligonucleotide, detector probes, and amplification primers, in a reaction mixture, e.g., in multi-state equilibrium, and that these values are useful in methods of maximizing amplification of rare allele sequences. The fraction of analyte, represented by “α” in various complexes within the reaction can be determined using the equations shown in FIG. 6, using the starting concentrations of amplification primer (P₀), blocker oligonucleotide (B₀), detector probe (D₀), and polymerase (E₀), and the respective equilibrium constants, K₁-K₅, for each of the different complexes, as discussed in connection with FIG. 5. FIG. 7 shows a model estimator for the number of amplicons, A_{n or B}ₙ, after n cycles, for two different targets (e.g., a wild-type target allele sequence and a rare mutant or rare variant target allele sequence), in a single reaction with limiting reagents (e.g., polymerase), calculated using the fraction of extendible complexes, “f e.,” determined using the equations shown in FIG. 6. The present embodiments are based, in part, upon the discovery that the fe. must be less than about 0.5, i.e., less than 0.4, 0.3, 0.2, 0.1, or less, for adequate blocking of amplification/detection of wild-type target allele sequences such that variant or mutant target allele sequences present in a sample at an initial copy number that is at 100-fold less (e.g., 200-fold, 300-fold, 400-fold, 500 fold, 600-fold, 700-fold, 800-fold, 900-fold, 1000-fold, 10000-fold or greater) than that of the wild-type target sequences.

### Kits

Aspects of the disclosure also relate to kits containing the reagents and compositions to carry out the methods described herein. Such a kit can comprise a carrier being compartmentalized to receive in close confinement therein one or more containers, such as tubes or vials. One of the containers may contain at least one unlabeled or detectably labeled primer or probe disclosed herein. The primers, including amplification primers, oligonucleotide blockers and detector probes can be present in dried form (e.g., lyophilized or other) or in an appropriate buffer as necessary. One or more containers may contain one or more enzymes or reagents to be utilized in PCR reactions. These enzymes may be present by themselves or in admixtures, in dried form or in appropriate buffers.

Finally, the kit can include all of the additional elements necessary to carry out the methods disclosed herein, such as buffers, extraction reagents, enzymes, pipettes, plates, nucleic acids, nucleoside triphosphates, filter paper, gel materials, transfer materials, autoradiography supplies, and the like.

The kits according to the present invention will comprise at least: (a) a blocker oligonucleotide, (b) a forward and reverse amplification primer, (c) an allele specific detector probe, and (d) instructions for using the provided amplification primer pair, blocker oligonucleotide, and allele specific detector probe.

In some embodiments, the kits include additional reagents that are required for or convenient and/or desirable to include in the reaction mixture prepared during the methods disclosed herein, where such reagents include: one or more polymerases; an aqueous buffer medium (either prepared or present in its constituent components, where one or more of the components may be premixed or all of the components may be separate), and the like. The various reagent components of the kits may be present in separate containers, or may all be pre-combined into a reagent mixture for combination with template nucleic acid.

In addition to the above components, in some embodiments, the kits can also include instructions for practicing the methods disclosed herein. These instructions can be present in the kits in a variety of forms, one or more of which may be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address that may be used via the internet to access the information at a removed site.

### EXAMPLES

The following examples are provided to demonstrate particular situations and settings in which this technology may be applied and are not intended to restrict the scope of the invention and the claims included in this disclosure.

### Example 1

The following example demonstrates that the methods disclosed herein can be used to effectively detect multiple rare variant target allele sequences in samples comprising an excess (100 fold or more) of wild-type or alternative variant or mutant target allele sequences.

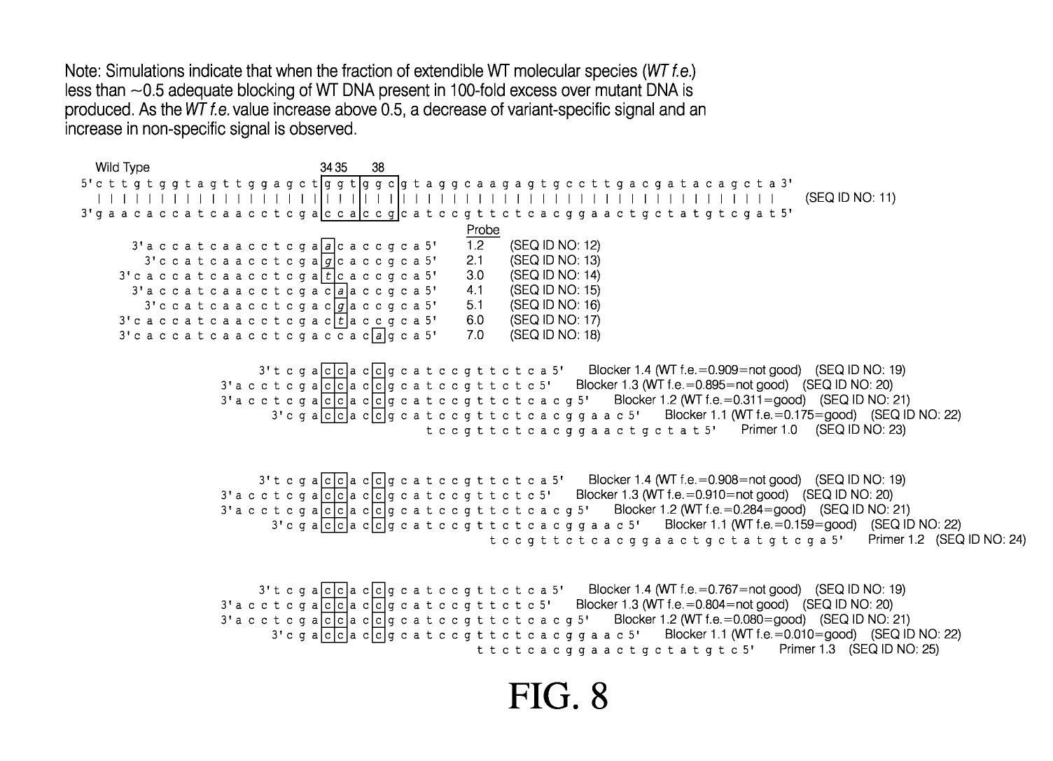

KRAS allelic variants G34T, G34C, G34A, and G38A, which are commonly used in the diagnosis prognosis of various cancers, as well as predicting the sensitivity of tumors to certain therapeutics, were used as an exemplary system to demonstrate the efficacy of the methods described herein. FIG. 8 shows the target region of interest in KRAS, including the wild-type sequence, as well as the position of the G34A, G34T and G38A variants.

Shown in FIG. 8 are three different amplification primers, i.e., Primer 1.0, Primer 1.2 and Primer 1.3 designed to amplify the target region of interest. Also shown are four different blocker oligonucleotides, i.e., blocker oligonucleotide 1.4, blocker oligonucleotide 1.3, blocker oligonucleotide 1.2 and blocker oligonucleotide 1.1 that include non-extendible 3′-OH modifications in accordance with the methods described above, and that are designed to preferentially binding to the wild-type target allele sequence compared to the various mutant allele sequences present at positions 34, 35, and 38 of KRAS, as shown in FIG. 8. Also shown are seven different detector probes, i.e., probes 1.2, 2.1, 3.0, 4.1, 5.1, 6.0 and 7.0 designed for the detection of G34A, wt, G34T, G35A, G35G, G35T and G38A alleles. The detector probes are configured to generate a detectable, fluorescent signal upon hybridization to target, measurable in real time.

Using the methods described herein above, the entropy, enthalpy, equilibrium constants, and fraction of each molecular species present at equilibrium were calculated as shown in FIGS. 5 and 6. These values were calculated for both wild-type and G34T DNA. Among the values calculated are the fraction of extendible molecular species (fe.) wild-type (WT fe.) and G34T (mutant fe.) DNA. The calculated values also include the fraction of analyte (either wild-type or G34T) bound to extendible species containing a detector probe(s) (represented by PADE in FIGS. 5 and 6). The PADE species produce target amplification and detectable signal during PCR, whereas the other extendible species (PAE and PAbE) produce amplification but not detectable signal. For reaction mixtures containing more than one detector probe, the fraction of analyte involved in each PADE species was calculated, and these values are used to estimate the signal produced by each respective probe. The various fe. values were used to perform PCR simulations in which the samples contained a 100-fold excess of wild-type target allele sequence compared to the G34T mutant allele sequence.

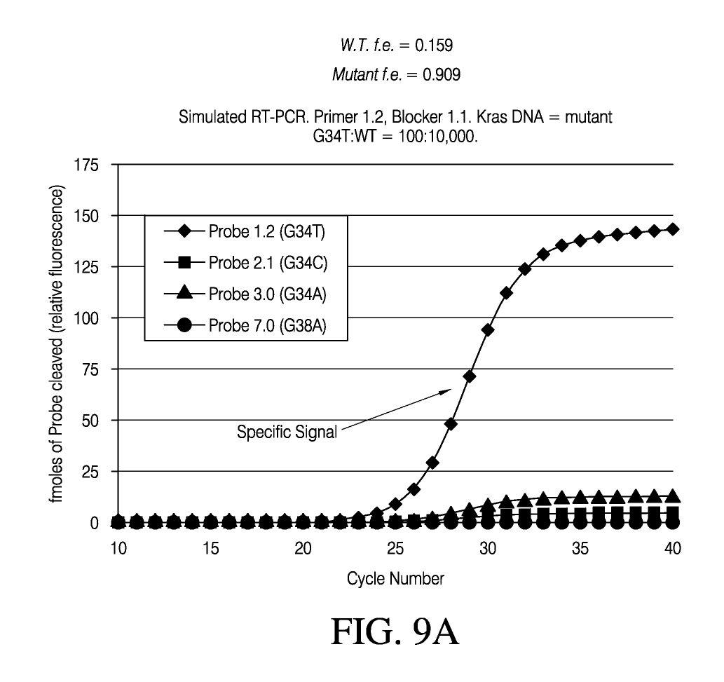

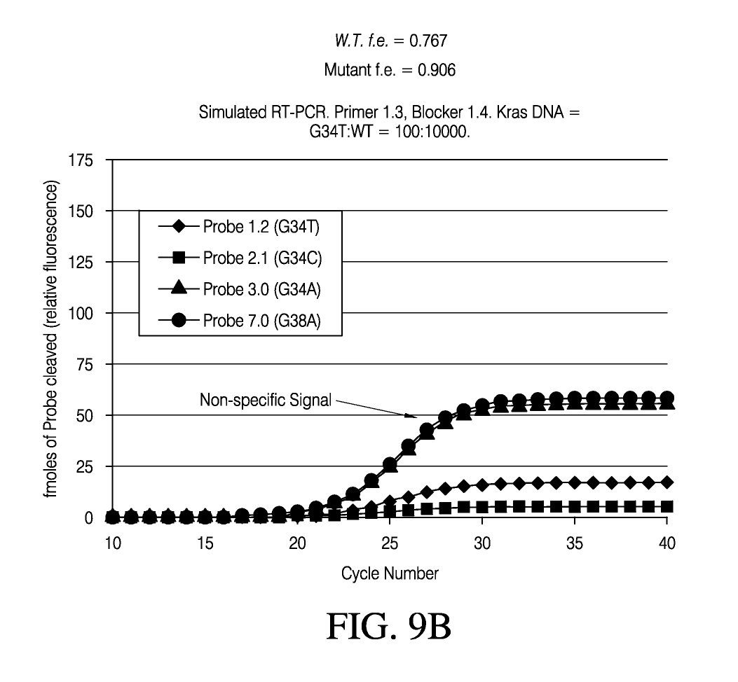

FIG. 9A shows the results of a simulated PCR reaction containing primer 1.2, blocker 1.1, and detector probes 1.2, 2.1, 3.0 and 7.0. As shown, a specific signal is detectable for G34T, whereas either very weak or no signal is produced from probes directed to the mutant target alleles not present in the sample. For this reaction, WT f.e. was 0.159 and mutant f.e. was 0.909, predictive of suppression of wild-type target amplification, but strong amplification of mutant target. In contrast, FIG. 9B shows the results of PCR simulation for reaction mixtures containing the same detector probes (1.2, 2.1, 3.0 and 7.0), but a different primer (primer 1.3) and blocker (blocker 1.4). Again, the wild-type allele is present in 100-fold excess over the mutant G34T allele. This primer-blocker combination results in calculated values for mutant fe. of 0.906, and WT f.e. of 0.767, the latter of which is predictive of significant amplification of both wild-type and mutant target alleles. As shown in FIG. 9B, only weak signal is produced for the probe directed at the G34T allele, while significantly stronger signals are produced from probes directed at mutant alleles not present in the reaction mixture. These non-specific signals are produced by hybridization of probes to wild-type DNA, which because of the insufficient suppression of amplification by the blocker 1.4, is present at much higher levels than the G34T allele throughout the course of the PCR reaction.

The foregoing data demonstrate that the methods disclosed herein can be used to effectively detect and identify rare mutant or variant target allele sequences against a background of excess wild-type sequences. The methods disclosed herein thus represent an extremely efficient, efficacious means to detect sequence polymorphisms and mutations that have wide-ranging clinical and experimental uses.

### Example 2

The following example demonstrates how the methods disclosed herein can be used to detect methyl cytosine residues in the death associated protein −1 (DAPK-1) promoter region. Changes in methylation status within the promoter region of DAKP-1 are frequently associated in with a variety of types of cancer and therefore accurate assessment of methylation patterns can be an important diagnostic indicator (Raval et al., (2007), Cell, 129: 879-890; Candiloro et al Epigenetics 2011 6: 500-507).

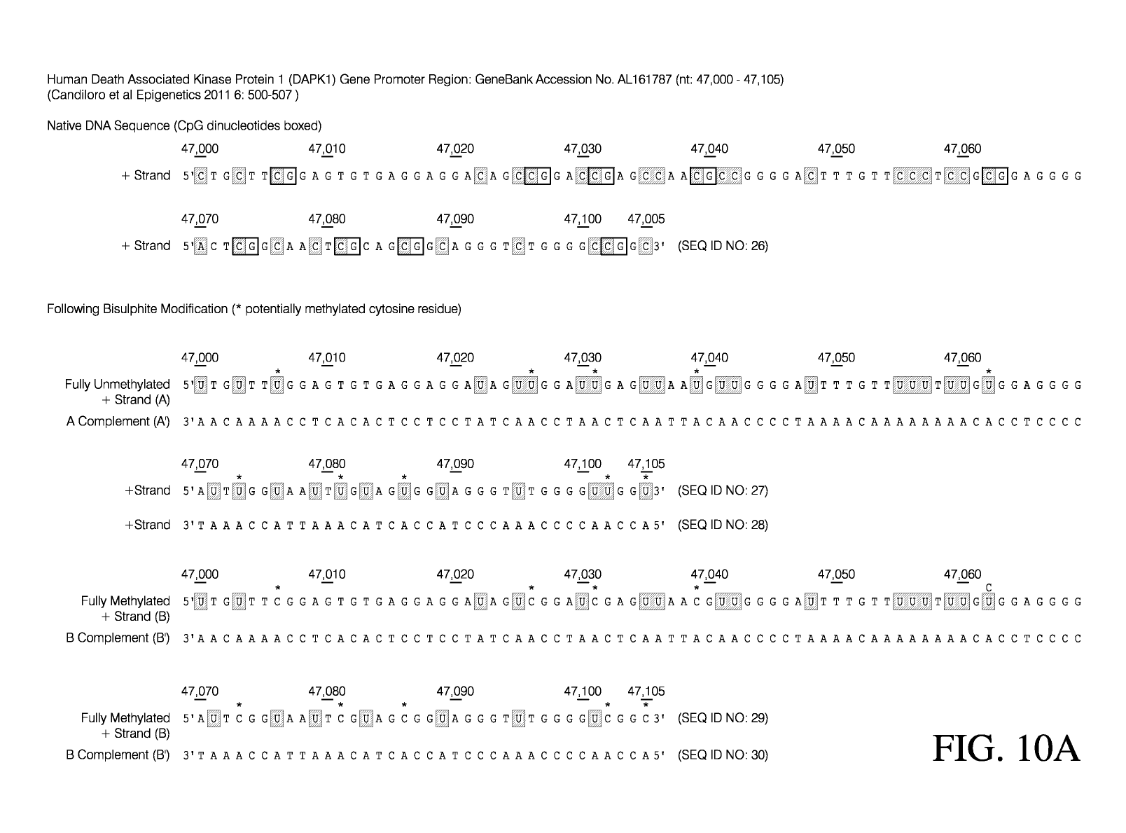

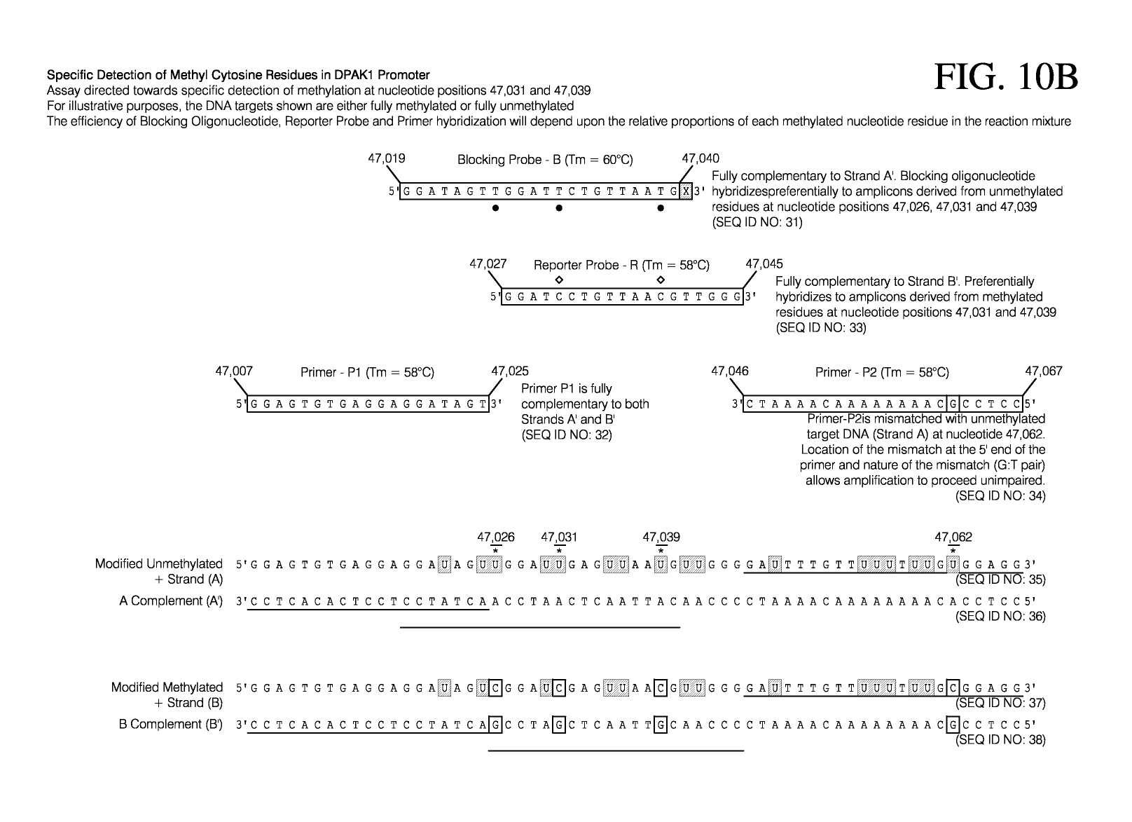

FIG. 10A shows a 105 bp target sequence within the promoter region of DAKP-1. CpG sites, which are often the sites of altered cytosine methylation patters, are shown in boxes. FIG. 10A also shows the unique sequences generated following treatment of the DAKP-1 promoter target sequence, when the sample DNA is originally fully unmethylated, or fully methylated. Specifically, as shown, there are nine cytosine residues that are potentially methylated, and that would be resistant to bisulphite treatment.

FIG. 10B shows a shorter, 6lbp region within the target sequence shown in FIG. 10A. As shown by the asterisks, four potential methylation sites, e.g., at nucleotide positions 47026, 47031, 47039 and 47062 exist within this region. Table 2 below illustrates the 16 possible DNA methylation patters within the DAPK-1 promoter region shown in FIG. 10B.

TABLE 2

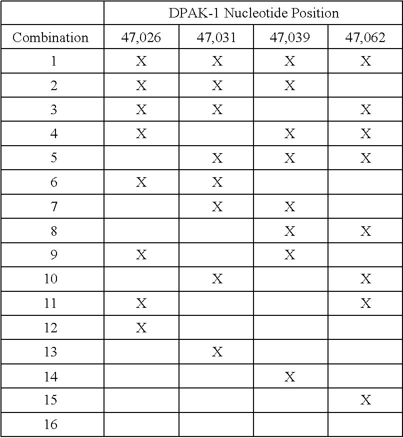

X: methyl cytosine residue;
Shaded box corresponds to residues detected by Reporter Probe-R

FIG. 10B illustrates how the use of methylation-specific amplification primers, methylation-specific reporter probes, and methylation-specific modulator oligonucleotides can be used to determine whether a sample comprising the DAPK-1 promoter target sequence comprises aberrant methylation. Primer P1 is fully complementary to sample DNA that is either fully methylated or unmethylated following modification with sodium bisulphite. By contrast, primer P2 includes a guanine residue that is mismatched with a converted uracil residue in the modified sample nucleic acids from a fully unmethylated sample, but which is complementary to modified sample nucleic acids from a fully methylated sample. Due to the fact that the mismatch is not at the 3′ end of the reverse primer, however, amplification can still occur under standard amplification conditions. The reporter probe R contains 2 cytosine residues that are mismatched with the modified sample nucleic acids from a fully unmethylated sample, but which are complementary to modified sample nucleic acids from a fully methylated sample. As such, the reporter probe preferentially hybridizes to the amplicon derived from sample nucleic acids that are methylated, compared to amplicons derived from sample nucleic acids that are unmethylated. Blocking probe B includes 3 thymine residues that hybridize to uracil residues present in the modified unmethylated sample, but that are mismatched with the guanine residues present in the modified methylated sample. Blocking probe contains a modification at its 3′ end that inhibits extension. As such, the blocking probe will preferentially hybridize to amplicons derived from the unmethylated sample nucleic acids, as compared to the methylated sample nucleic acids. Accordingly, using primers P1, P2, reporter probe R, and blocking oligonucleotide B, on can preferentially amplify and detect rare methylated sample nucleic acids, e.g., within a sample comprising an abundance of unmethylated nucleic acids.

The embodiments described and claimed herein is not to be limited in scope by the specific embodiments herein disclosed, since these embodiments are intended as illustrations of several aspects of the invention. Any equivalent embodiments are intended within the scope of this invention. Indeed, various modifications of the embodiments in addition to those shown and described herein will become apparent to those skilled in the art from the foregoing description. The appended claims are intended to cover such modifications.

What is claimed is:

1. A method to detect a first variant target sequence in a sample comprising nucleic acids, the method comprising:
providing the biological sample;
contacting the biological sample with:
a pair of amplification primers comprising a forward primer and a reverse primer, said pair of amplification primers configured to amplify a target amplicon, wherein said amplicon comprises a wild-type target sequence or a variant target sequence, and wherein the pair of amplification primers amplifies both wild-type target sequences and variant target sequences;
a blocking primer that preferentially hybridizes to the wild type target sequence compared to a first variant target sequence under amplification conditions; and
a reporter probe, wherein said reporter probe comprises an oligonucleotide that preferentially hybridizes to the first variant target sequence compared to the wild-type target sequence under amplification conditions; wherein said contacting takes place under amplification conditions; and
measuring the hybridization of the reporter probe to the first variant target sequence, wherein hybridization of the reporter probe to the first variant target sequence produces a detectable signal indicative of the presence or amount of first variant target species in the biological sample.

2. The method of claim 1, wherein hybridization of the blocking primer to the amplicon comprising the variant target sequence creates an extendible species, and wherein hybridization of the blocking primer to the wild type target sequence creates a non-extendible species, and wherein a fraction of extendible species (f.e.) represents the fraction of extendible species of a total number target amplicons.

3. The method of claim 2, wherein the fie. is less than about 0.5.

4. The method of any of the preceding claims, wherein the biological sample comprises about 100-fold excess of wild-type target sequences compared to variant target sequence.

5. The method of any of the preceding claims, further comprising detecting a second variant target sequence, wherein the blocking primer preferentially hybridizes to the wild type target sequence compared to the second variant target sequence under amplification conditions, wherein said method further comprises:
contacting the biological sample with a second reporter probe, wherein said second reporter probe comprises an oligonucleotide that preferentially hybridizes to the second variant target sequence compared to the wild-type target sequence under amplification conditions; wherein said contacting takes place under amplification conditions; and
measuring the hybridization of the second reporter probe to the second variant target sequence, wherein hybridization of the reporter probe to the second variant target sequence produces a detectable signal indicative of the presence or amount of second variant target species in the biological sample.

6. The method of claim 5, wherein the biological sample is simultaneously contacted with the first reporter probe and the second reporter probe.

7. The method of any of the preceding claims, wherein the first reporter probe comprises a modified nucleic acid.

8. The method of any of the preceding claims, wherein the first variant target sequence is in a gene selected from the group consisting of: KRAS, BRAF, EGFR, TP53, JAK2, NPM1, and PCA3.

9. The method of any of the preceding claims, wherein the second variant target sequence is in a gene selected from the group consisting of: KRAS, BRAF, EGFR, TP53, JAK2, NPM1, and PCA3.

10. The method of any of the preceding claims, wherein the method comprises performing real-time PCR.

11. The method of any of the preceding claims, wherein the method comprises performing isothermal amplification.

12. The method of any of the preceding claims, wherein the blocking primer is between 15 and 30 nucleotides in length.

13. The method of any of the preceding claims, wherein the first reporter probe is between 15 and 30 nucleotides in length.

14. The method of any of the preceding claims, wherein the blocking probe is longer than the first reporter probe.

15. The method of any of the preceding claims, wherein the first reporter probe does not overlap with either the forward or reverse amplification primer.

16. The method of any of the preceding claims, wherein the first reporter probe overlaps with the blocker oligonucleotide, wherein the overlap between the first reporter probe and the blocker oligonucleotide does not extend to the 3′ end of the reporter probe.

17. The method of any of the preceding claims, wherein the first reporter probe overlaps with the blocker oligonucleotide, wherein the overlap between the first reporter probe and the blocker oligonucleotide does not extend to the 5′ end of the blocker oligonucleotide.

18. The method of claim 15, wherein the overlap between the first reporter probe and the blocker oligonucleotide does not extend to the 5′ end of the blocker oligonucleotide.

19. The method of any of the preceding claims, wherein the blocker oligonucleotide overlaps with either the forward or reverse amplification primer, and wherein the overlap does not extend to the 3′ end of the blocker oligonucleotide.

20. The method of claim 18, wherein the overlap between the blocker oligonucleotide and the forward or reverse amplification primer does not extend to the 5′ end of the forward or reverse amplification primer.

21. The method of any of the preceding claims, wherein the first reporter probe is selected from the group consisting of a TAQMAN® reporter probe, a SCORPION® reporter probe, a hybridization (FRET) probe, and a molecular beacon probe.

22. A method of detecting the presence of a methylated cytosine residue in a target DNA sequence in a sample, comprising:
treating the sample with a reagent that specifically modifies unmethylated cytosine residues to uracil residues to generate a modified sample DNA to generate a modified sample DNA target sequence;
combining the modified sample DNA target sequence with an amplification primer pair comprising a forward primer and a reverse primer, wherein the forward and reverse amplification primers are fully complementary to modified sample DNA that comprises methylated cytosines, and that is not fully complementary to modified sample DNA that comprises uracil residues to create an amplification reaction mixture;
contacting the reaction mixture with a reporter probe that is fully complementary to target amplicons generated from modified sample DNA that comprises methylated cytosines, and that is not fully complementary to target amplicons generated from modified sample DNA that comprises uracil;
subjecting the reaction mixture to an amplification reaction to generate target amplicons;
detecting the amount of reporter probe bound to target amplicons produced from the amplification reaction.

23. The method of claim 22, wherein the reaction mixture further comprises a blocking probe that competes with both the reverse primer and the reporter probe for hybridizing to the amplified target sequence, wherein the blocking probe preferentially hybridizes to amplicons produced form modified sample DNA that comprises uracil residues.
